(19) United States
(12) Patent Application Publication
(10) Pub. No.: US 2006/0229735 A1
(43) Pub. Date: Oct. 12, 2006

(54) Device for tissue engineering

(75) Inventors: Shuvo Roy, Shaker Heights, OH (US); George F. Muschler, Cleveland Heights, OH (US); Aaron J. Fleischman, University Heights, OH (US); Alvaro Mata, Chicago, IL (US)

Correspondence Address: TAROLLI, SUNDHEIM, COVELL & TUMMINO L.L.P., 1300 EAST NINTH STREET, SUITE 1700, CLEVEVLAND, OH, 44114, US

(73) Assignee: The Cleveland Clinic Foundation

(21) Appl. No.: 11/250,287

(22) Filed: Oct. 14, 2005

Related U.S. Application Data
(60) Provisional application No. 60/619,121, filed on Oct. 15, 2004.

(51) Int. Cl. A61F 2/02; C12N 5/08

(52) U.S. Cl. 623/23.74; 435/396

(57) ABSTRACT

An implant includes a plurality of parallel layers spaced apart by a plurality of members. Each layer has a substantially uniform thickness between opposite surfaces and a plurality of openings that permit fluid flow through an interior of the implant defined by the layers. The surfaces of each layer includes an array of micro-structures. Each micro-structure has a substantially uniform shape and an average height of about 1 nm to about 20 μm.

### RELATED APPLICATIONS

The present application claims priority from U.S. Provisional Application No. 60/619,121 filed Oct. 15, 1004, herein incorporated by reference in its entirety.

### TECHNICAL FIELD

The present invention generally relates to an apparatus for tissue regeneration, and, more particularly, to an implantable scaffold for tissue regeneration.

### BACKGROUND OF THE INVENTION

Tissue engineers are attempting to engineer virtually every human tissue. Potential tissue-engineered products include cartilage, bone, heart valves, nerves, muscle, bladder, liver, etc. Tissue engineering techniques generally require the use of a porous scaffold that can serve as a three-dimensional (3D) template for initial cell attachment and subsequent tissue formation both in vitro and in vivo. The scaffold can provide support for cells to attach, proliferate, and maintain their differentiated function. The scaffold's architecture ultimately defines the shape of the newly grown soft or hard tissue.

Clinically established materials such as collagen and polyglycolide have been considered to be the preferred material for scaffolds. Unfortunately, commercial scaffolds made of these materials fail to facilitate adequate cell attachment and proliferation. The surfaces of commercially available scaffolds are smooth, and the smooth scaffold surfaces simply do not provide a reliable surface upon which potential colonizing cells can adhere. Furthermore, production of commercially available scaffolds is hampered by generally slow manufacturing techniques and, more specifically, a lack of control over the fabrication process.

The challenge for more advanced scaffold systems is to arrange cells/tissue in an appropriate 3D configuration and present molecular signals in an appropriate spatial and temporal fashion so that the individual cells will grow and form the desired tissue structures. Accordingly, a need exists for improved 3D scaffolds which can not only be produced economically and on a large scale, but which can also promote a desired tissue formation response.

### SUMMARY OF THE INVENTION

The present invention relates to an implant that comprises a plurality of parallel layers spaced apart by a plurality of members. The layer can have a substantially uniform thickness between opposite surfaces and a plurality of openings that permit fluid flow through an interior of the implant defined by the layers. The surfaces of each layer can include an array of micro-structures. Each micro-structure can have a substantially uniform shape and an average height of about 1 nm to about 20 μm.

The present invention also relates to a scaffold for tissue engineering applications. The scaffold includes a plurality of parallel layers spaced apart by a plurality of members. Each layer of the scaffold can have a substantially uniform thickness between opposite surfaces and a plurality of openings that permit fluid flow through an interior of the scaffold defined by the layers. The surfaces of each layer can include an array of micro-structures. Each micro-structure can have a substantially uniform shape and an average height of about 1 μm to about 20 μm.

The present invention further relates to a method of forming an implant for tissue engineering. In the method, a plurality of layers can be provided. Each layer can have a substantially uniform thickness between opposite surfaces. A plurality of members can extend from the layers. A plurality of openings can extend through the layers. The surfaces of each layer can include an array of micro-structures. Each micro-structure can have a substantially uniform shape and an average height of about 1 nm to about 20 μm. The layers can be bonded together so that the layers are substantially parallel to and separated from each other by the plurality of members.

### BRIEF DESCRIPTION OF THE DRAWINGS

The foregoing and other features of the present invention will become apparent to those skilled in the art to which the present invention relates upon reading the following description with reference to the accompanying drawings, in which:

FIG. 1 illustrates a schematic view of a scaffold of an implantable device in accordance with an aspect of the invention.

FIG. 2 illustrates a schematic sectional view of a portion of the scaffold of an implantable device in accordance with an aspect of the invention.

FIG. 3 illustrates a schematic sectional view of a portion of the scaffold of an implantable device in accordance with another aspect of the invention.

FIG. 4 illustrates a first photoresist layer being patterned in accordance with a method of forming a scaffold in accordance with an aspect of the invention.

FIG. 5 illustrates a second photoresist layer being patterned in accordance with the method of the invention.

FIG. 6 illustrates a first photoresist layer being patterned in accordance with a method of the invention.

FIG. 7 illustrates a first mold portion used in the method of forming a scaffold in accordance with an aspect of the invention.

FIG. 8 illustrates a fourth photoresist layer being patterned in accordance with the method of the invention.

FIG. 9 illustrates a second mold portion used in the method of the invention.

FIG. 10 illustrates a layer of uncured PDMS being provided between the first mold portion and the second mold portion in accordance with the method of the invention.

FIG. 11 illustrates the layer of PDMS being compressed between the first mold portion and the second mold portion in accordance with the method of the invention.

FIG. 12 illustrates a patterned film layer being released from the first mold portion and the second mold portion in accordance with the method of the invention.

FIG. 13 Illustrates a top plant view along lines 13-13 of the patterned film layer of FIG. 12.

FIG. 14 illustrates a cross-sectional view of the patterned film layer along lines 14-14 of FIG. 13.

FIG. 15 illustrates the patterned release layer being provided with an adhesive in accordance with the method of the invention.

FIG. 16 is a cross-sectional view of a plurality of stacked patterned film layers in accordance with the method of the invention.

FIG. 17 illustrates differences in colony (fluorescent microscope) and cell (SEM) morphology after fixation and staining. (a) On smooth surfaces, colonies were circular in shape and cells had broaden and flattened shape; (b) on posts, colonies were bigger and cells had narrower and longer processes; and (c) on channels, both colonies and cells were highly aligned along the direction of the channels.

FIG. 18 illustrates SEM images of (a) cells growing on 10 μm posts with long processes (white arrows and inset) directed towards posts and other cells; and (b) a closer view demonstrating cells with highly contoured morphologies and processes directed towards posts and their neighbor cells.

FIG. 19 illustrates cells on 5 μm posts tended to grow in an orthogonal manner (a); and between and along the array of posts (b), forming a channel-like alignment.

FIG. 20 illustrates cells on channels aligned in the direction of the channels (black arrows), while cells on smooth regions kept their random orientation (white arrows) (a). The majority of cells aligned within the channels (b); while some aligned on the top of the ridges (c). In some cases distortions of the ridges at the points of cell contact were observed (black arrows), which suggest a strong attachment (d).

FIG. 21 illustrates fluorescent microscope images depict (a) actin cytoskeleton being aligned along the direction of the channels; and (b) actin cytoskeleton having a more random orientation on smooth surfaces.

FIG. 22 illustrates fluorescent microscope images depict cells growing on 10 μm posts showing (a) focal adhesion contacts (green spots shown by white arrows) and cell nuclei (blue); and (b) actin cytoskeleton (red) and cell nuclei.

FIG. 23 is a graph illustrating quantification of height from the bottom of the lowest observed actin microfilaments to the bottom of the nucleus (bars with strips), and from the lowest actin to the top of the nucleus (solid bars). The difference between the two bars for each substrate corresponds to the cell nucleus height. N corresponds to the number of cells measured on each substrate.

FIG. 24 illustrates time-lapse microscopy depicting a cell (white arrow) at the edge of a growing colony that is migrating within a region with 10 μm posts (a), reaches the smooth area (b), and as it moves into the smooth (c), it attaches to the posts (d, e, f) and brings itself back towards the post filled region (g, h). Time scale is 10 hrs.

FIG. 25 illustrates phase contrast (a) and fluorescent microscope (b) of a colony of cells (stained with DAPI) preferentially growing within 10 μm posts and avoiding the smooth areas.

FIG. 26 illustrates time-lapse microscopy depicting transfected cells growing within 10 μm posts. Notice the fluorescent actin cytoskeleton (white arrow) being attached to a post (white circle for clarity) (a); detaching the actin from the post as it migrates (b); and finally releasing the post (c). Time scale is 15 min.

FIG. 27 illustrates time-lapse microscopy depicting a cell (white arrow) at the interface between channels and smooth surfaces. The cell spreads perpendicular to the channels (a, b); attaches to the ridge of one of the channels (c); and propels itself inside of the channel while aligning its body within them (d-f). Time scale is 4 hrs.

FIG. 28 are graphs illustrating a summary of quantification for (a) cell number/colony and (b) cell number from five fields of vision. Both quantifications exhibited a similar trend with posts having higher number of cells, with the highest number for the 10 μm POSTS (*: p<0.05).

FIG. 29 illustrates immunofluroscent microscope top-views of cells growing on (a, b) 3D PDMS Texture scaffolds, which consistently showed bigger colonies than cells on (c, d) 3D PDMS Smooth scaffolds; and (e) cells growing on a 3D PDMS Textures scaffold exhibiting a well-defined actin cytoskeleton (red) located within and around the posts. Scale bar on a−d=400 μm.

FIG. 30 illustrates a three-dimensional reconstruction of confocal images taken on the cells that migrated up on to the 3D PDMS Texture Scaffold demonstrating (a) a top-view of cells growing on a horizontal surface around the 200 μm column that was in contact with the tissue culture dish (solid arrow) and the 300 μm pores around the column (broken arrow), and a (b) side-view of the same region depicting cells growing on the 200 μm column (solid arrow), to the horizontal surface, and up the walls of the 300 μm pores (broken arrow).

FIG. 31 is a graph illustrating the mean cell number/colony quantification from all three experiments on both 3D PDMS Texture (top inset image) and 3D PDMS Smooth (bottom inset image) scaffolds. Cells growing on 3D PDMS Texture scaffolds consistently formed bigger colonies than cells on the 3D PDMS Smooth scaffolds on all three experiments.

### DETAILED DESCRIPTION

The present invention relates to a device that can be used for tissue engineering applications, such as for generating tissue, bone, and cartilage, as well as for the formation of micro-fluidic devices, and/or lab-on-chip systems. The device comprises a scaffold with a micro-architecture that includes a plurality of interconnected pores in the micron range and a plurality of micro-textured surfaces. By micro-architecture, it is meant the overall structure and features of the scaffold, which is on the order of micro-meters (μm) or millimeters (mm). The micro-architecture of the scaffold can include the shape, geometry, and orientation of supports, pores, and channels of the scaffold.

By micro-textures or micro-textured, it is meant the surface structures, features or convolutions of the surfaces of the scaffold. By micro-textured surface, it is meant a surface or portion of a surface having a multiplicity of microtextures over the majority of its extent, for example over 70% or more of its extent, and over 90% or more of its extent, which may or may not also comprise occasional larger features.

The overall micro-architecture and micro-texture of the scaffold is such that the device functions to allow the continued flow of dissolved nutrients in biological or biocompatible fluids into, through, and around the device. The device includes a geometry, pore size, and surface micro-texture that are conducive to cell migration, attachment, growth, differentiation, and proliferation. These features can be manipulated to selectively populate a particular region(s) of the device with different cell types, or to allow ingrowth into one region, while promoting cell attachment and proliferation in another. In this way, the device can facilitate, for example, the regeneration of the complex tissue and supporting tissue interfaces. As demonstrated by the examples, these devices can be engineered to allow and encourage growth of connective tissue progenitor cells.

Channels bounded by supports and layers and consisting of passageways of defined width, length, and orientation are a micro-architectural feature of the devices described herein. Staggered channels extending through the device and offset in different layers of the device are another feature of the device. Staggering the channel and walls increases the strength of the device relative to a straight through channel design. The width of the channels can range from about 100 μm to about 900 μm s, (e.g. about 200 μm to about 500 μm), in order to maximize the surface area available for cell seeding without compromising structural integrity or homogeneity of tissue formation.

In addition, the channels facilitate the transport of nutrients to the cells and removal of cellular by-products and material degradation by-products which all may occur whether the device is colonized by cells before or after implantation in the body. The micro-architecture of the device allows cells (e.g., connective tissue progenitors) to contact the device throughout the thickness of the device not only superficially. This is important due to the limited migration capacity of cells, such as connective tissue progenitors.

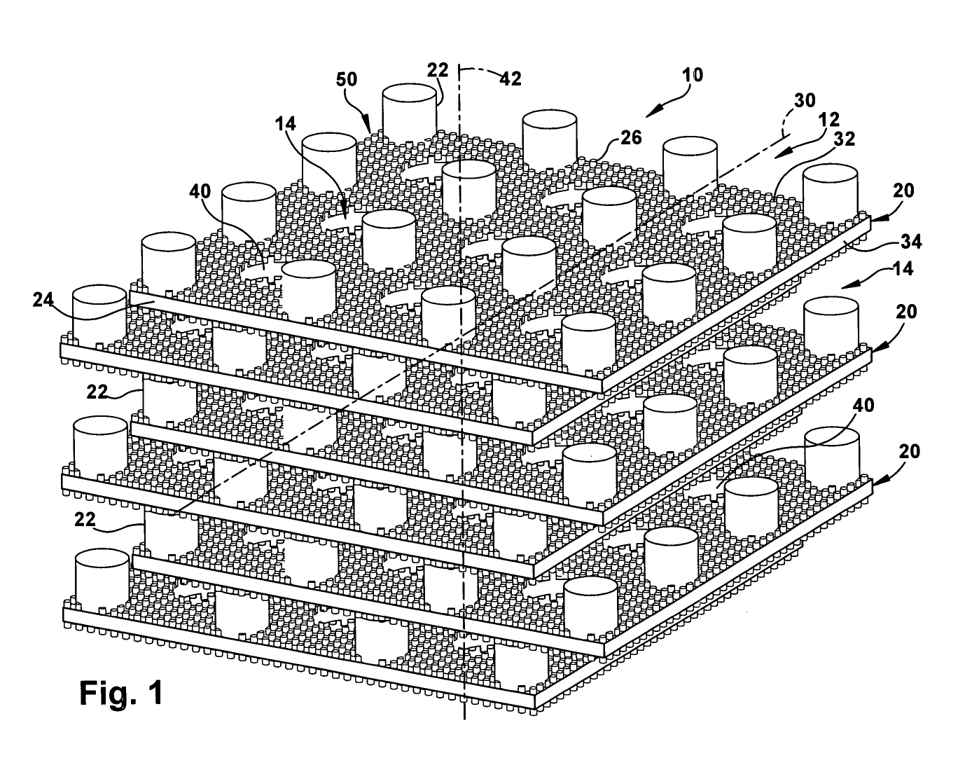

FIG. 1 is a schematic illustration of a device 10 in accordance with an aspect of the invention. The device 10 can be implantable for in vivo applications in a subject being treated and/or used in in vitro applications for tissue engineering and/or culturing. The device 10 comprises a scaffold 12 with a microarchitecture that has a plurality of interconnected pores 14. The pores 14 provide the scaffold 12 with a porosity of, for example, at least about 60% by volume of the scaffold 12 (e.g., at least about 65% by volume of the scaffold 12). A porosity of at least about 60% by volume provides an “open cell” environment in the scaffold 12, which is important to encourage cell or tissue growth throughout the scaffold 12, i.e., three-dimensional cell or tissue growth.

The features of the present device 10 make it particularly suitable for use in tissue engineering and more notably, cell transplantation, because it provides a biocompatible scaffold that cells can colonize in a three-dimensional manner via the interconnected porous network of the scaffold 12. This is significant when considering the transplantation of any cells that yield tissues, especially those requiring neoangiogenesis, such as bone tissue. Moreover, when used for cell transplantation, the scaffold 12 is biodegradable, the degradation of which can be controlled such that cell growth may be simultaneous with the degradation of the scaffold 12.

The scaffold 12 includes a plurality of substantially parallel layers 20 that are spaced apart (or separated) by a plurality of columns 22. Each layer 20 extends from a first end 24 to a second end 26 and parallel to an axis 30. Each layer 20 can have a substantially uniform thickness between opposite surfaces 32 and 34 of the layer 20. The thickness of each layer 20 can be for example, about 5 μm to about 150 μm. The layers 20 can each have substantially the same thickness or different thicknesses depending on the application of the device 10.

Each layer 20 includes a plurality of openings 40 (or through holes) that permit fluid flow through an interior of the device 10 defined by the layers 20. The openings 40 can extend substantially parallel to axis 42 and substantially normal to the surfaces 32 and 34 of the layers 20. The openings 40 can be provided in each layer 20 in substantially parallel rows, which extend parallel to the axis 30. The distance between each opening 40 in the rows can be substantially equal. In an aspect of the invention, the distance can be essentially equal to the diameter of the openings 40. Adjacent rows of the openings 40 can spaced from each other at about the same distance which the openings 40 are spaced in respective rows so that a substantially uniform array of openings 40 is provided in each layer 20. The array of openings 40 of each layer 20 can be offset from the array of openings 40 in adjacent layers so that the scaffold 12 includes a plurality of meandering channels or pores 14 that extend normal to the layers 20.

Each opening 40 can have a substantially circular shape and a diameter to allow the continued flow of fluids into the interior and through the device 10. The size and number of openings 40 can also be provided to, for example, readily promote cell migration, attachment, growth, differentiation, and proliferation in tissue engineering applications. The diameter of each opening 40 in the layers 20 can be, for example about 100 μm to about 900 μm. In another aspect of the invention, the openings 40 can have an average diameter of about 200 μm to about 500 μm (e.g. about 300 μm).

It will be appreciated that although the openings 40 in the layers 20 are substantially circular, the openings 40 can have other shapes, including uniform shapes and substantially regular polygonal shapes, without departing from the scope of the present invention. Further, although the openings 40 are illustrated as being substantially equal in size, openings 40 with varying sizes can be provided in the layers 20 as long as the intended function of the device 10 is not adversely affected.

The columns (or supports) 22 used to separate the layers 20 can extend substantially normal from opposite facing surfaces 32 and 34 of adjacent layers 20. The columns 22 can have a substantially uniform height (or length) such that adjacent layers 20 are parallel to and separated from each other by substantially the same distance (i.e., the height of the columns 22).

Each column 22 can have a substantially cylindrical shape and a diameter to allow the continued flow of fluids through the device 10. The height and width of the columns 22 as well as the number of columns 22 and their respective placement between each layer 20 can be selected to allow the continued flow of fluids into the interior of and through the device 10 and to, for example, readily promote cell migration, attachment, growth, differentiation, and proliferation in tissue engineering applications.

By way of example the average height of the columns 22 can be, for example, about 100 μm to about 500 μm (e.g., about 200 μm) so that adjacent layers 20 can be separated from each other by about 100 μm to about 500 μm (e.g., about 200 μm). The width of the columns 22 can be about 100 μm to about 500 μm (e.g., about 200 μm).

It will be appreciated that although the columns 22 provided between the layers 20 are substantially cylindrical, the columns 22 can have other shapes, including uniform shapes and substantially regular polygonal shapes, without departing from the scope of the present invention.

The columns 22 can be arranged between each layer in substantially parallel rows that extend parallel to the axis 30. The distance between each column 22 in the rows can be substantially equal. In an aspect of the invention the distance can be twice the average diameter of the columns 22. Adjacent rows of the columns 22 can spaced from each other at about the same distance which the columns 22 are spaced in respective rows so that a substantially uniform array of columns 22 is provided between adjacent layers 20. The rows of columns 22 can also be arranged between rows of openings 40 so that each column 22 extends from a portion of the surface 32 or 34 of each layer 20 between spaced apart openings 40 in the layer 20.

The surfaces 32 and 34 of each layer 20 include a surface microtexture that is provided by an array of micro-structures 50. The micro-structures 50 can enhance cell migration, attachment, growth, differentiation, and proliferation on the surfaces 32 and 34 of the layers 20.

The micro-structures 50 can be oriented generally uniformly in relation to the surfaces 32 and 34 of the layers 20. The micro-structures 50 can be oriented normal to the surfaces 32 or 34, the surface normal direction being defined as that direction of a line perpendicular to an imaginary plane lying tangent to the surface 32 or 34 at a point of contact of the base of the micro-structure 50 with the surface 32 or 34. The surface normal direction is seen to follow the contours of the surface 32 or 34 of the layer 20. The major axes of the micro-structures 50 can be parallel to each other.

The micro-structures 50 can be of uniform height and shape, and have uniform cross-sectional dimensions along their major axes. The height of each micro-structure 50 can be that height effective to promote cell attachment, migration, adhesion, differentiation, and proliferation. This height can be about 1 nm to about 20 μm. By way of example, each micro-structure 50 can have an average height of about 1 μm to about 10 μm.

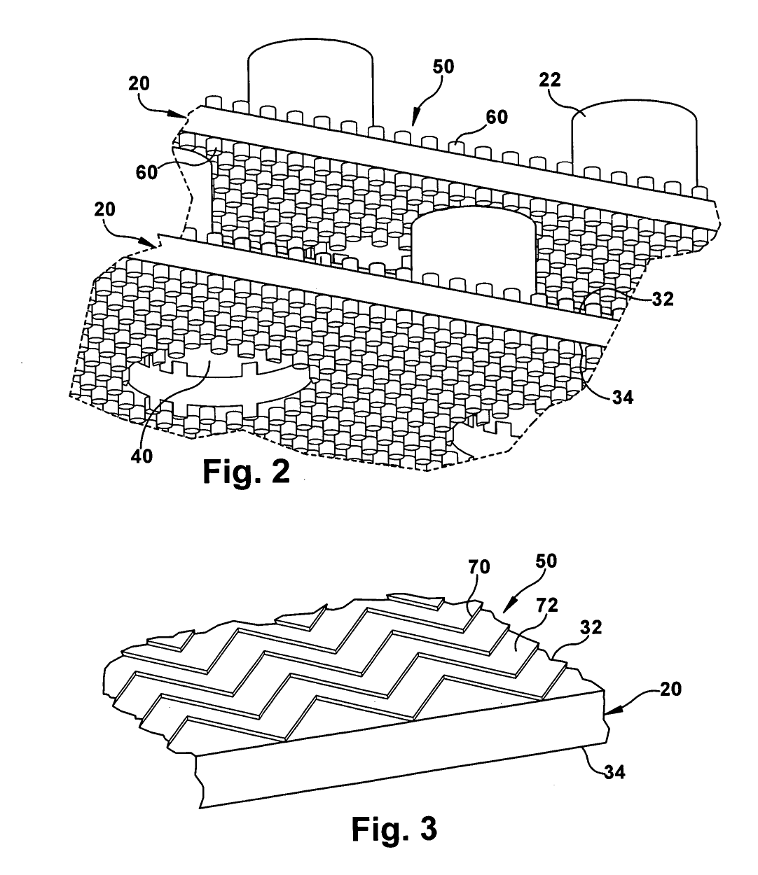

In one aspect of the invention, as illustrated in FIG. 2, the micro-structures 50 can comprise a plurality of posts 60 that extend substantially normal to the surfaces 32 and 34 of each layer 20. The posts 60 can be substantially cylindrical in shape and have substantially uniform heights and substantially uniform diameter. By way of example, the posts 60 can have an average height of about 1 μm to about 10 μm and an average diameter of about 5 μm to about 50 μm (e.g., about 10 μm).

It will be appreciated that although the posts 60 on the surfaces 32 and 34 of the layers 20 are substantially cylindrical, the posts can have other shapes, including uniform shapes and substantially regular polygonal shapes, without departing from the scope of the present invention.

The posts 60 can be arranged on the surfaces 32 and 34 of each layer 20 in substantially parallel rows, which extend parallel to the axis 30 (FIG. 1). The distance between each post 60 in the rows can be substantially equal. In an aspect of the invention, the distance can be substantially equal to the diameter of the post 60 diameter. Adjacent rows of the post 60 can be spaced from each other at about the same distance which the posts are spaced in respective rows so that a substantially uniform array of posts 60 is provided in on the surfaces 32 and 34 of each layer 20.

The posts 60 can have an areal coverage about 60% to about 80% of the horizontal surface area of the layers 20. This areal coverage can be readily modified by changing the orientation, sizes, and shapes of the posts 60.

It has been found that colonies of cells cultured on the posts 60 exhibited higher cell number and density than colonies of cells cultured on smooth surfaces. Additionally, colonies of cells cultured on about 10 μm posts exhibited higher cell number than any other post size texture, and a significant increase in cell number (e.g., 442%) compared to colonies cultured on smooth (e.g., 71%) surfaces.

In another aspect of the invention, as illustrated in FIG. 3, the micro-structures 50 can comprise a plurality of ridges 70 that extend substantially normal to the surfaces 32 (and 34) of each layer 20. The ridges 70 on surfaces 32 and 34 of each layer 20 can have substantially uniform heights and extend substantially parallel to one another so as to form a plurality of channels 72 on the surfaces 32 and 34 of the layers 20. By way of example, the ridges 70 can have an average height of about of about 1 nm to about 15 μm (e.g., about 11 μm) and an average width of about 1 nm to about 10 μm (e.g., about 5 μm). Adjacent parallel ridges 70 can be separated from each other by about 10 μm to about 50 μm (e.g., about 45 μm) so as to form channels 72 having an average width of about 10 μm to about 50 μm (e.g., about 45 μm).

The channels 72 formed by the ridges 70 can be arranged on the surfaces 32 and 34 of each layer 20 in substantially parallel rows. Colonies of cells cultured on the surfaces 32 and 34 of layers 20 provided with such channels 72 tend to be highly aligned and have an enhanced cell density along the direction of the channels 72. This can be advantageous where it is desirable to direct the migration and cell density of cells cultured on the surfaces 32 and 34 of the layers 30.

It will be appreciated that the micro-structures 50 can have a variety of orientations as well as straight and curved shapes, (e.g., rods, cones, pyramids, cylinders, laths, and the like that can be twisted, curved, or straight), and any one layer 20 can comprise a combination of orientations and shapes.

The scaffold 12 of the device 10 in accordance with the present invention can be manufactured using natural or synthetic structural materials that have inherent ability to encourage cell attachment and provide mechanical integrity in terms of tensile strength and compressibility. The materials can include resorbable and/or non-resorbable materials.

The materials used in the manufacture of the devices 10 described herein can be biocompatible and bioresorbable over periods of weeks or longer, and generally encourage cell attachment. The term “bioresorbable” is used herein to mean that the material degrades into components which may be resorbed by the body and which may be further biodegradable. Biodegradable materials are capable of being degraded by active biological processes such as enzymatic cleavage.

Examples of such materials can include biocompatible polymers and osteoconductive materials. Biocompatible polymers that can potentially be used to form the scaffolds in accordance with the present invention can include poly(lactide), poly(lactide-co-glycolide) (PLGA) of varying ratios, polystyrene, poly(glycolide), poly(acrylate)s, poly(methyl methacrylate), poly(hydroxyethyl methacrylate), poly(vinyl alcohol), poly(carbonate), poly(ethylene-co-vinyl acetate), poly(anhydride), poly(ethylene), poly(propylene), poly(hydroxybutyrate), poly(hydroxyvalerate), poly(urethane)s, poly(ether urethane), poly(ester urethane), poly(arylate), poly(imide), poly(anhydride-co-imide), poly(aminoacids), poly(phosphazene), and polydimethylsiloxane (PDMS).

Natural polymers, which can potentially be used include polysaccharides, such as cellulose, dextrans, chitin, chitosan, glycosaminoglycans; hyaluronic acid or esters, chondroitin sulfate, and heparin; and natural or synthetic proteins or proteinoids, such as elastin, collagen, agarose, calcium alginate, fibronectin, fibrin, laminin, gelatin, albumin, casein, silk protein, proteoglycans, Prolastin, Pronectin, or BetaSilk. Mixtures of any combination of polymers may also be used.

In an aspect of the invention the polymer can comprise a polydimethylsiloxane (PDMS), such as Sylgard 184, which is commercial available from Dow Corning, Inc. Midland, Mich. Polydimethylsiloxanes can be advantageously employed to form the scaffold 12 of the present invention as it is biocompatible, highly inert, simple to manufacture, substantially transparent, and capable of readily being processed to form micron sized features.

In another aspect of the invention, the polymer can comprise a polylactic acid/polyglycolic acid (PLA/PLGA) polymer. Polylactic acid/polyglycolic acid polymers are biodegradable and can be potentially processed into scaffolds.

Examples of osteoconductive materials, which can potentially be used in forming a scaffold 12 in accordance with the invention include ceramics, such as hydroxyapatite (HA), tricalcium phosphate (TCP), calcium phosphate, calcium sulfate, alumina, bioactive glasses and glass-ceramics, animal derived structural proteins, such as bovine collagen, and demineralized bone matrix processed from human cadaver bone. Commercially available materials include: ProOsteon 500 (Interpore International), BoneSource (Orthofix) and OSTEOSET (Wright Medical Technology), Grafton Gel, Flex, and Putty (Osteotech), and Collagraft (Zimmer).

Optiontally, the device 10 may be modified in order to enhance its properties for use as a scaffold 12 for cellular growth. Modifications typically effecting an apparatus used as a support for cellular growth can also be suitable to modify the present scaffold 12. Such modifications function to enhance biological response and can include surface chemistry modifiers or biological factors or growth factors, which can be releasable in a physiological environment for the purpose of stimulating cell attachment, growth, maturation, and differentiation in the area of the device. Bioactive agents that can be directly dissolved in a biocompatible solvent can be advantageously employed. Examples of bioactive agents include proteins and peptides, polysaccharides, nucleic acids, lipids, and non-protein organic and inorganic compounds, referred to herein as “bioactive agents” unless specifically stated otherwise. These materials have biological effects such as growth factors, differentiation factors, steroid hormones, cytokines, lymphokines, antibiotics, and angiogenesis promoting or inhibiting factors.

It is also possible to incorporate materials not exerting a biological effect such as air, radiopaque materials, such as barium, or other imaging agents for the purpose of monitoring the device 10 in vivo.

In order to promote cell attachment, cell adhesion factors, such as laminin, pronectin, or fibronectin or fragments thereof, e.g. arginine-glycine-aspartate, may be coated on or attached to the device 10. The device 10 may also be coated or have incorporated cytokines or other releasable cell stimulating factors such as; basic fibroblast growth factor (bFGF), transforming growth factor beta (TGF-beta), nerve growth factor (NGF), insulin-like growth factor-1 (IGF-1), growth hormone (GH), multiplication stimulating activity (MSA), cartilage derived factor (CDF), bone morphogenic proteins (BMPs) or other osteogenic factors, anti-angiogenesis factors (angiostatin),

In addition, either exogenously added cells or exogenously added factors including genes may be added to the device 10 before or after its placement in the body. Such cells include autografted cells which are derived from the patients tissue and have (optionally) been expanded in number by culturing ex vivo for a period of time before being reintroduced. For example, cartilage tissue may be harvested and the cells disaggregated therefrom, and cultured to provide a source of new cartilage cells for seeding the devices. The devices may also be seeded with cells ex vivo and placed in the body with live cells attached thereto.

DNA of a gene sequence, or portion thereof, coding for a growth factor or other of the auxiliary factors mentioned above may also be incorporated into the device 10 or added to the device 10 before or after placement in the body. The DNA sequence may be “naked” or present in a vector or otherwise encapsulated or protected. The DNA sequence may also represent an antisense sequence of a gene or portion thereof.

There are essentially no limitations on the bioactive agents that can be incorporated into the device 10. Those materials which can be processed into particles using spray drying, atomization, grinding, or other standard methodology, or those materials which can be formed into emulsions, microparticles, liposomes, or other small particles, and which remain stable chemically and retain biological activity in a polymeric matrix, can be employed.

The scaffold of the implantable device in accordance with the present invention can be made using soft lithographic microfabrication techniques. In accordance with an aspect of the invention, the microfabrication techniques can include dual-side molding polymer films with a film mold to form thin three dimensionally shaped and microtextured film layers. The film molds can be fabricated using photolithography methods. The molded films can comprise features, such as through holes or openings and columns as well as arrays of smaller micro-structures distributed on the top and bottom surfaces of the film. The molded films can then be aligned, stacked, and bound together to achieve the porous scaffold in accordance with the present invention. The scaffold so formed may be in a final form, which is suitable for cell colonization and implantation or may be used as a negative mold for another implantable material (e.g., calcium phosphate) which will constitute the scaffold.

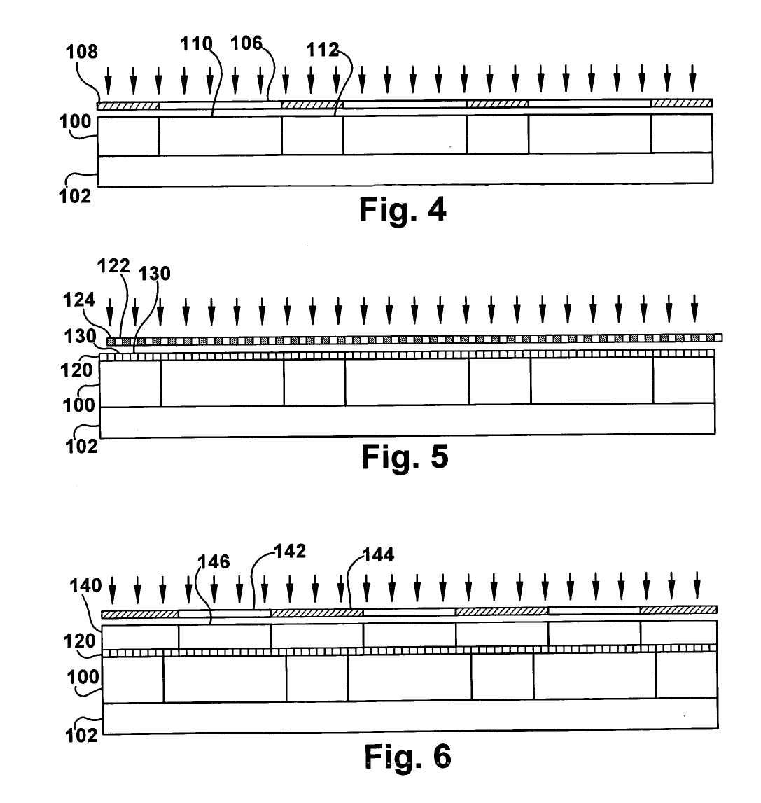

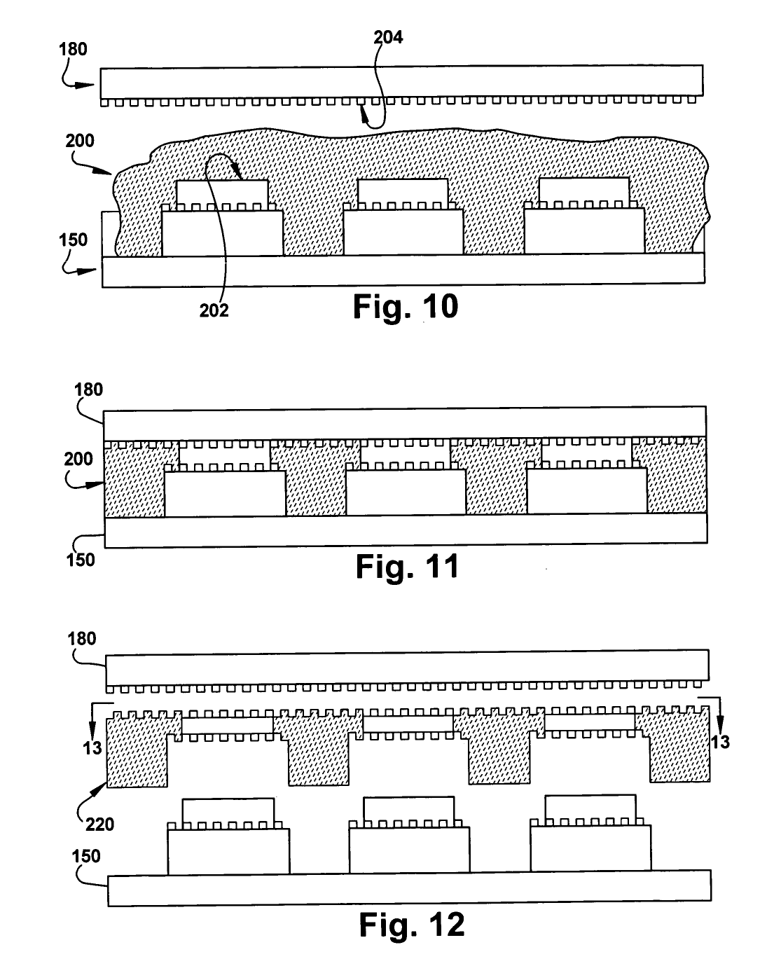

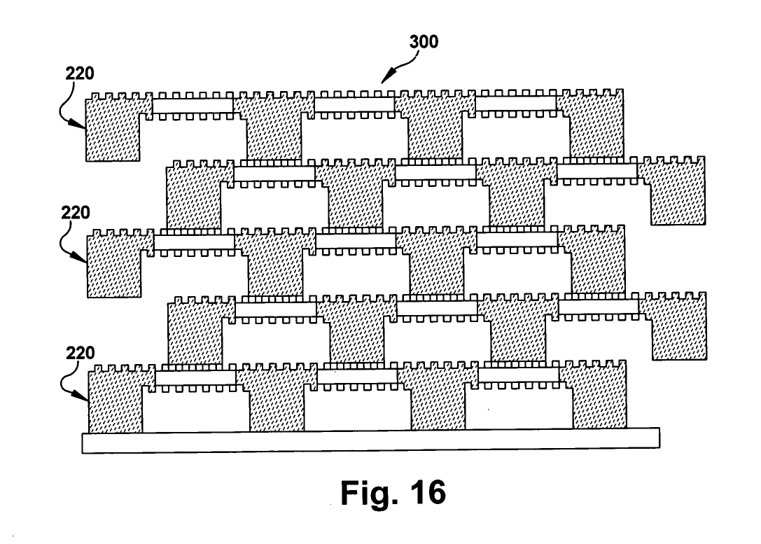

FIGS. 4-16 illustrate a methodology of fabricating a scaffold of an implantable device in accordance with an aspect of the present invention. In the methodology, a first mold portion is formed (FIGS. 4-7), a second mold portion is formed (FIGS. 8-9), a film is molded with the first mold portion and the second mold portion (FIGS. 10-15), and molded films are bonded to form a scaffold of an implantable device (FIGS. 15-16).

Referring to FIG. 4, a first photoresist layer 100 is formed over a substrate 102 and patterned. The substrate 102 can comprise an underlayer material, such as a single crystal silicon or epitaxial silicon or wafer. By way of example, the substrate 102 can comprise a standard 100 mm-diameter, 500 μm thick, n-type (100)-oriented silicon wafer. It will be appreciated that other substrate materials typically used in microfabrication techniques can also be used to form the substrate 102.

The first photoresist layer 100 can comprise a SU-8 photoresist (MicroChem Corp., Newton, Mass.) material that is provided on the substrate layer 102 via conventional photoresist coating techniques (e.g., spin coating). The photoresist layer 100 can have a thickness, for example, of about 100 μm to about 300 μm (e.g., about 200 μm) and be soft baked in a C-005 convection oven (Lindberg/Blue M, Asheville, N.C.) at a temperature and for an amount of time effective to at least partially cure the first photoresist layer 100 (e.g., about 95° C. for about 55 minutes) prior to patterning. It will be appreciated that the first photoresist layer 100 can include other photoresist materials used in microfabrication techniques and that these photoresist materials can be provided on the substrate 102 via conventional spin-coating or spin casting deposition techniques.

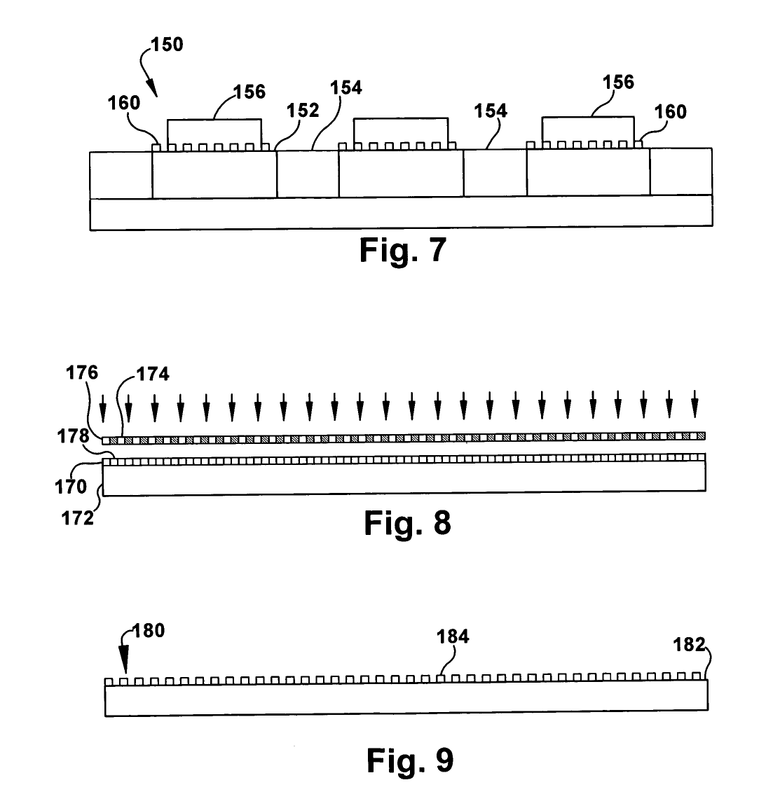

The first photoresist layer 100 can be patterned by exposing the first photoresist 100 layer through openings 106 in a mask 108 at a wavelength and energy (e.g., about 365 nm, at about 375 mJ/cm²) effective to cure exposed portions 110 of the first photoresist layer 100. The exposed portions 110 of the first photoresist layers 100 can correspond to areas of a first mold portion (FIG. 7) that define a substantially planar first surface of a layer used to form a scaffold in accordance with the present invention. The unexposed portions 112 of the first photoresist layers 100 can correspond to areas of a first mold portion (FIG. 7) that define columns extending from the first surface of the layer (FIG. 14) used to form a scaffold (FIG. 16) in accordance with the present invention. Following exposure, the first photoresist layer 100 can be post exposure baked at a temperature and time (e.g., about 95° C., about 25 min) effective to further cure the exposed portions 110 of the first photoresist layer 100.

FIG. 5 illustrates a second photoresist layer 120 is formed over the first photoresist layer 100 and patterned. The second photoresist layer 120, like the first photoresist layer, can comprise an SU-8 photoresist (MicroChem Corp., Newton, Mass.) material. The second photoresist layer 120 can be provided on the first photoresist layer 100 via conventional photoresist coating techniques (e.g., spin coating or spin casting).

The second photoresist layer 120 can have a thickness, for example, of about 1 μm to about 25 μm (e.g., about 10 μm) and be soft baked in a in a C-005 convection oven (Lindberg/Blue M, Asheville, N.C.) at a temperature of about 95° C. for about 5 minutes (min) prior to patterning. It will be appreciated that the second photoresist layer 120, like the first photoresist layer 100, can include other photoresist materials used in microfabrication techniques and that these photoresist materials can be provided on the first photoresist layer 100 via conventional spin-coating or spin casting deposition techniques.

The second photoresist layer 120 can be patterned by exposing the second photoresist layer 120 through openings 122 in a second mask 124 at a second wavelength and energy (e.g., about 365 nm, at about about 100 mJ/cm²) effective to at least partially cure exposed portions 130 of the second photoresist layer 120. The exposed portions 130 of the second photoresist layer 120 can correspond to shapes on the first mold portion (FIG. 7) that define the micro-structures on the first surface of the layer (FIG. 14) used to form a scaffold (FIG. 16) in accordance with the present invention. Following exposure, the second photoresist layer 120 can be post exposure baked at a temperature and time (e.g., about 95° C., about 5 min) effective to further cure the exposed portions 130 of the second photoresist layer 120.

FIG. 6 illustrates a third photoresist layer 140 is formed over the second photoresist layer 130 and patterned. The third photoresist layer 140, like the first photoresist layer 100 and the second photoresist layer 120, can comprise an SU-8 photoresist (MicroChem Corp., Newton, Mass.) material. The third photoresist layer 140 can be provided on the second photoresist layer 120 via conventional photoresist coating techniques (e.g., spin coating or spin casting).

The third photoresist layer 140 can have a thickness, for example, of about 50 μm to about 200 μm (e.g., about 100 μm) and be soft baked in a C-005 convection oven (Lindberg/Blue M, Asheville, N.C.) at a temperature of about 95° C. for about 5 minutes prior to patterning. It will be appreciated that the third photoresist layer 140, like the first photoresist layer 100 and the second photoresist layer 120, can include other photoresist materials used in microfabrication techniques and that these photoresist materials can be provided on the second photoresist layer 120 via conventional spin-coating or spin casting deposition techniques.

The third photoresist layer 140 can be patterned by exposing the third photoresist layer 140 through openings 142 in a mask 144 at a wavelength and energy (e.g., about 365 nm, at about about 375 mJ/cm²) effective to at least partially cure exposed portions 146 of the third photoresist layer 140. The exposed portions 146 of the third photoresist layer 140 can correspond to areas of the first mold (FIG. 7) that define through holes in the layer (FIG. 14) used to form a scaffold (FIG. 16) in accordance with the present invention. Following exposure, the third photoresist layer 140 can be post exposure baked at a temperature and time (e.g., 95° C., 5 min) effective to further cure the exposed portions of the third photoresist layer 140.

FIG. 7 illustrates that following patterning of the third photoresist layer 140, the first photoresist layer 100, the second photoresist layer 120, the third photoresist layer 140 are developed to form a first mold portion 150 (i.e., male mold portion) of a mold. The photoresist layers 100, 120, 140 can be developed, for example, simultaneously using an SU-8 Developer (MicroChem Corp.) (25° C., 50 min) with agitation.

The first mold portion 150 simultaneously developed from the photoresist layers 100, 120, and 140 can comprises a multilevel SU-8 mold that includes a substantially planar surface 152 with a plurality of holes 154 that extend substantially normal to the surfaces 152. The holes can be provided in the surface 152 in substantially parallel rows. The distance between each hole 154 in the rows can be substantially equal. The adjacent rows of the holes 154 can be spaced from each other at about the same distance which the holes 154 are spaced in respective rows so that a substantially uniform array of holes 154 is provided in the surface 152 of the first mold portion 150. Each hole 154 can have a substantially cylindrical shape and a diameter of, for example, about 100 μm to about 900 μm.

A plurality of columns 156 extend substantially normal from the surface 152 of the first mold portion 150. Each column 156 can have a substantially uniform height (or length) (e.g., about 100 μm) and a substantially cylindrical shape with a substantially uniform diameter (e.g., about 300 μm). The columns 156 can be arranged on the surface 152 of the first mold portion 150 in substantially parallel rows. The distance between each column 156 in the rows can be substantially equal. Adjacent rows of the columns 156 can spaced from each other at about the same distance which the columns 156 are spaced in respective rows so that a substantially uniform array of columns 156 extends from the surface 152 of the first mold portion 150.

The surface 152 of the first mold portion 150 includes a surface 160 microtexture that is provided by an array of micro-structures 160. The micro-structures can be oriented generally uniformly in relation to the surface 152 of the first mold portion 150. The micro-structures 160 can comprise a plurality of posts 160 that extend substantially normal to the surface 152 of the first mold portion 150. The posts 160 can be substantially cylindrical in shape and have substantially uniform heights and substantially uniform diameters. By way of example, the posts can have an average height of about 7 μm to about 10 μm and an average diameter, for example, of 10 μm.

The posts 160 can be arranged on the surface 152 in substantially parallel rows. The distance between each post 160 in the rows can be substantially equal. Adjacent rows of the posts 160 can spaced from each other at about the same distance which the posts are spaced in respective rows so that a substantially uniform array of posts 160 is provided in on the surface 152 of the firs mold portion 150.

FIG. 8 illustrate that a fourth photoresist layer 170 is formed over a second structure layer 172 and patterned. The second substrate 172 can comprise an under layer material, such as a single crystal silicon or epitaxial silicon or wafer. By way of example, the substrate 172 can comprise a standard 100 mm-diameter, 500 μm thick, n-type (100)-oriented silicon wafer.

The fourth photoresist layer 170 can comprise a SU-8 photoresist (MicroChem Corp., Newton, Mass.) material that is provided on the substrate 172 via conventional photoresist coating techniques (e.g., spin coating). The fourth photoresist layer 170 can have a thickness of, for example, about 1 μm to about 25 μm (e.g., about 10 μm) and be soft baked in a C-005 convection oven (Lindberg/Blue M, Asheville, N.C.) at a temperature of about 95° C. for about 5 minutes prior to patterning. It will be appreciated that the fourth photoresist layer 170 can include other photoresist materials used in microfabrication techniques and that these photoresist materials can be provided on the substrate 172 via conventional spin-coating or spin casting deposition techniques.

The fourth photoresist layer 170 can be patterned by exposing the fourth photoresist layer 170 through openings 174 in a mask 176 at a wavelength and energy (e.g., about 365 nm, at about 100 mJ/cm²) effective to at least partially cure exposed portions 178 of the fourth photoresist layer 170. The exposed portions 170 of the fourth photoresist layers can correspond to areas of a second mold portion (FIG. 9) that define micro-structures on a second surface of the layer (FIG. 14) used to form a scaffold (FIG. 16) in accordance with the present invention. Following exposure, the fourth photoresist layer 170 can be post exposure baked at a temperature and time effective (e.g., about 95° C., about 5 min) to further cure the exposed portions 178 of the fourth photoresist layer 170.

Following patterning of the fourth photoresist layer 170, the fourth photoresist layer 170 is developed to form a microtextured surface (not shown). The fourth photoresist layer can be developed, for example, using an SU-8 Developer (MicroChem Corp.) (25° C., 12 min) with agitation. The microtextured surface developed from the fourth photoresist layer 170 comprises a substantially planar surface with a plurality of micro-structures (i.e., about 10 μm deep, about 10 μm diameter posts). This micro-textured surface can then used to cast and realize a transparent polymer (e.g., PDMS) into second mold portion 180 (i.e., female mold portion) (FIG. 9). The second mold portion 180 comprises a substantially planar surface 182 with a plurality of micro-structures 184 (i.e., 10 μm deep, 10 μm diameter posts). This second mold portion 180 was made of PDMS because its transparency and flexibility facilitate alignment and subsequent separation of the two mold portions 150 and 180 during dual-sided molding of the layer.

Following formation of the second mold portion 180 and the first mold portion 150, mold surfaces of the second mold portion 180 and the first mold portion 150 can be optionally coated (not shown) with a mold release agent, such as 1H,1H,2H,2H-Perfluorodecyltrichlorosilane (Lancaster, Pelham, N.H.). The mold release agent can facilitate release of a polymer molded by the first mold portion 150 and the second mold potion 180.

FIG. 10 illustrates a layer of a readily molded biocompatible uncured polymer 200 is provided between the first mold portion 150 and the second mold portion 180. The biocompatible uncured polymer 200 can comprise, for example, uncured polydimethylsiloxane (PDMS). PDMS is biocompatible, highly inert, simple to manufacture, substantially transparent, and capable of being readily processed to form micron sized features. The uncured polymer 200 can be poured over the mold surfaces 202 and 204 of, respectively, mold portions 150 and 180, distributed to cover all the patterned areas of the mold portions 150 and 180, and then degassed (e.g., for about 15 min).

FIG. 11 illustrates the first mold portion 150 and the second mold portion 180 are then aligned and brought into contact, with the patterned mold surfaces 202 and 204 facing each other, while squeezing the uncured polymer 200. The first mold portion 150 and the second mold portion 180 can be aligned, for example, by placing the first mold potion 150 and the second mold portion 180 in a custom mechanical jig (not shown), which allows horizontal, vertical, and rotational motion control for alignment of the mold portions 150 and 180 within about 10 μm. The first mold portion 150 and the second mold portion 180 can then placed inside of an oven heated at a temperature and an amount of time effective to cure the polymer 200 (e.g., at about 75° C. for about 2 hours to cure PDMS).

FIG. 12 illustrates that after curing, the first mold portion 150 and the second mold portion 180 are allowed to cool to room temperature and immersed in methanol to remove (or separate) both mold portions 150 and 180 from the squeezed polymer. The patterned film layer 220 so formed from the squeezed and cured polymer can then cut with a blade into, for example, about 1.5 cm by about 1.5 cm specimens.

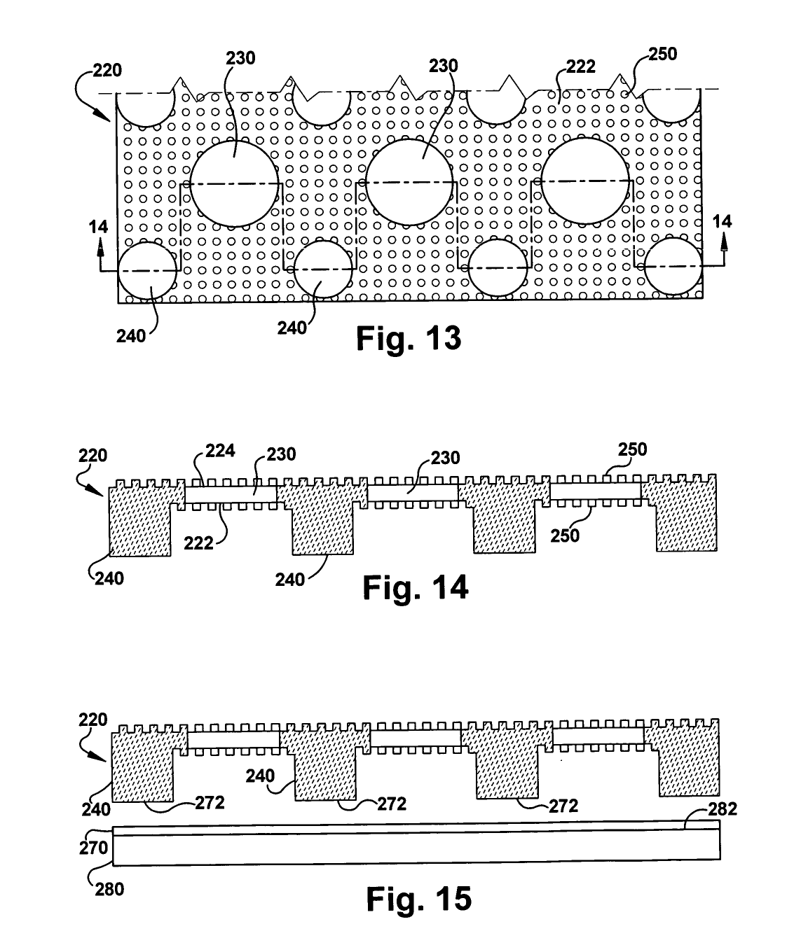

FIG. 13 and FIG. 14 illustrate respectively a top plan view of a portion of the patterned film layer 220 of FIG. 12, so formed, along lines 13-13 and a cross-sectional view of the patterned film layer 220 along lines 15-15. The patterned film layer 220 comprises a substantially planar first surface 222 and an opposite facing substantially planar second surface 224.

The patterned film layer 220 includes a plurality of openings 230 (or through holes) that extend substantially normal to the surfaces 222 and 224 of the patterned film layer 220. The openings 230 can be provided in the patterned film layer 220 in substantially parallel rows. The distance between each opening 230 in the rows can be substantially equal. Adjacent rows of the openings 230 can spaced from each other at about the same distance which the openings 230 are spaced in respective rows so that a substantially uniform array of openings 230 is provided in the patterned film layer 200. Each opening can have a substantially circular shape and a diameter of, for example, about 300 μm.

A plurality of columns 240 extend substantially normal from the first surface 222 of the patterned film layer 220. Each column 240 can have a substantially uniform height (or length) (e.g., about 200 μm) and a substantially cylindrical shape with a substantially uniform diameter (e.g., about 200 μm). The columns 240 can be arranged on the patterned film layer 230 in substantially parallel rows. The distance between each column 240 in the rows can be substantially equal. Adjacent rows of the columns 240 can spaced from each other at about the same distance which the columns 240 are spaced in respective rows so that a substantially uniform array of columns 240 extends from the patterned film layer 220.

The first surface 222 and the second surface 224 of the patterned film layer 220 include a surface microtexture that is provided by an array of micro-structures 250. The micro-structures 250 can enhance cell migration, attachment, growth, differentiation, and proliferation on the surfaces 222 and 224 of the patterned film layer 220. The micro-structures 250 can be oriented generally uniformly in relation to the surfaces 222 and 224 of the patterned film layer 220. The micro-structures 250 can comprise a plurality of posts 250 that extend substantially normal to the surface 222 and 224 of the patterned film layer 220. The posts 250 can be substantially cylindrical in shape and have substantially uniform heights and substantially uniform diameter. By way of example, the posts can have an average height of about of about 7 μm to about 10 μm and an average diameter, for example, of 10 μm.

The posts 250 can be arranged on the first surface 222 and the second surface 224 of the patterned film layer 230 in substantially parallel rows. The distance between each post 250 in the rows can be substantially equal. Adjacent rows of the posts can spaced from each other at about the same distance which the posts are spaced in respective rows so that a substantially uniform array of posts 250 is provided in on the surfaces 222 and 224 of the patterned film layer 220.

FIG. 15 illustrates that an adhesive 270 is provided on the ends 272 of the columns 240 of the patterned film layer 220. Providing an adhesive 270 on the ends 272 of the columns 240 allows patterned film layers 220 in accordance with the invention to be adhered together once stacked. In an aspect of the invention, the adhesive 270 can comprise uncured PDMS. The uncured PDMS can be provided on a substrate 280, such as a smooth 100 mm diameter, 500 μm thick, n-type (100)-oriented silicon wafer, by pouring the adhesive 270 on about a substantial portion of the substrate 280. The adhesive 270 can then be spin coated across a surface 282 of the substrate 280 (using, for example, a 400 Lite spinner (Laurell Technologies, North Wales, Pa.) at 4000 revolutions per minute (rpm)) to achieve an about 10 μm thick layer of the adhesive 270. The patterned film layer 280 is stamped on top of the uncured PDMS, so that the tips 272 of the columns 240 are wetted with uncured PDMS.

FIG. 16 illustrates that a plurality of patterned film layers 220 that include the adhesive coated column tips are stacked and aligned. The stacked and aligned patterned film layers 220 can then baked, for example at about 95° C. for about 30 minutes, to cure the PDMS (adhesive) and bond the patterned film layers. The bound patterned film layers 220 can form a scaffold 300 substantially similar to the scaffold illustrated in FIGS. 1-3.

The scaffold so formed can be used as the implantable device or as a negative mold for another implantable material that will constitute the scaffold. For example, where the scaffold is used as a negative mold, a suspension of calcium phosphate can be poured into the scaffold and allowed to set or cure. If PDMS is the polymer used to form the scaffold, the negative mold of PDMS can then be removed by sintering the scaffold and suspension in a high temperature furnace. The sintering process burns off the PDMS and leaves behind a calcium phosphate ceramic scaffold with a precisely defined macro- and micro-architecture surface containing micro-textured features. The fabrication of the negative PDMS mold can use multiple levels (up to 6) of SU-8 photoresists, which are processed in a single developing step.

In an aspect of the invention, a method of culturing cells for three-dimensional growth can be provided utilizing the scaffolds, so formed. The interconnected porous structure of the scaffold is especially suitable for tissue engineering, and notably bone tissue engineering. In the method, the scaffold can be seeded with cells, such as connective tissue progenitor cells, using conventional methods, which are well known to those of skill in the art. Cells, such as connective tissue progenitor cells, that are seeded onto the scaffold are then cultured under suitable growth conditions. The cultures are fed with media appropriate to establish the growth thereof.

The cells that are seeded on the scaffold and grown can comprise various types. More precisely, cell types includes connective tissue progenitor cells, hematopoietic or mesenchymal stem cells, as well as any other cells that can potentially yield cardiovascular, muscular, or any connective tissue. Cells may be of human or other animal origin. However, the scaffold of the present invention is particularly suited for the growth of connective tissue progenitor cells, especially cells that elaborate bone matrix. For tissue engineering, the cells may be of any origin. The cells are advantageously of human origin. The present method of growing cells in a three dimensional scaffold according to the invention allows seeded connective tissue progenitor cells, for example, to migrate through the scaffold to elaborate bone matrix, during the in vitro stage, with pervasive distribution in the structure of the scaffold.

The seeded scaffold can be used as an implantable device to generate biological structures. The micro-architecture and surface micro-textures can modulate tissue and bone formation by promoting cellular adherence and growth. To this end, the instant method provides scaffolds with precise micro-architectures and surface micro-textures which may enhance the attachment, migration, and proliferation of certain progenitor cells thereto.

It will be appreciated that, while for many human or animal applications, such as bone or tissue replacement, the scaffold is biocompatible and non toxic, it can also be biodegradable. However, it will be appreciated that in some of these applications it may be preferred or advantageous to use a biocompatible polymer which is not biodegradable in situations where a permanent scaffold is needed to support other tissue. Further, it will be appreciated that for non biological applications the requirements for biocompatibility and biodegradable need not be invoked.

The present invention also provides additional methods for using the scaffold. For instance, the scaffold can be used for bone marrow transplantation where, for example, bone marrow stem cells from a donor subject may loaded into a scaffold and then implanted into the bone of a recipient subject. Additionally, a scaffold may be used for delivery of therapeutically engineered marrow stem cells (e.g., transfected or genetically modified cells) to a bodily site of interest. Further, scaffolds in a accordance with the present invention may be loaded with various other progenitor cells may to create cartilage, muscle, and bio-artificial organs. Additionally, the methods of the present invention may can be employed to create highly compact, 3D structures for any number of micro-electro-mechanical systems, including, but not limited to, microfluidic devices, lab-on-chip systems, and high-throughput drug screening applications.

### EXAMPLES

The present invention is further illustrated by the following series of examples. The examples are provided for illustration and are not to be construed as limiting the scope or content of the invention in any way.

### Example 1

Effect of Surface Micro-Textures on Connective Tissue Progenitor Cells (CTPs)

Successful bone healing requires the presence of a sufficient number of CTPs to adhere, proliferate, and organize extracellular matrix molecules into a functional tissue. The state of the art approach of combining CTPs with a 3D scaffold to enhance regeneration in bone grafting procedures, involves rapid concentration and selection of this cell population from the bone marrow into the graft using selective attachment to a matrix surface. In vivo, this approach has been demonstrated to improve graft efficacy.

In vitro, CTPs can be assayed and cultured under specific biochemical conditions that promote osteoblastic differentiation. A single CTP from a fresh bone marrow sample gives rise to one colony of osteoblasts. The number of cells in the colony formed can provide an assay of proliferation, and the distribution of cells within a colony provides an indirect assay of relative migration.

At the cell-surface interface, both an appropriate physicochemical environment and an idoneous surface topography profoundly affect the overall behavior of the engineered tissue. The generation of surface topographies at the cellular level influences cell shape and modifies gene activity. Consequently, the incorporation of micro- and nanoscale topographies at the cell-substrate interface might provide an attractive approach to enhancing specific cell behaviors without destabilizing the delicate biochemical environment.

To address the need for precise and controlled cell guidance, there has been increased interest in the application of microfabrication and micromachining technologies to tissue engineering. Although initial exploration of these technologies for biomedical applications had been focused on the development of diagnostic tools, recent studies have increasingly concentrated on creating model systems to study cellular behavior. Microfabrication technology closely parallels the multidimensional size scale of living cells, and therefore can be exploited to provide tissue engineering scaffolds that possess topographical, spatial, and chemical properties to optimize control over cell behavior.

To increase the understanding of CTP-surface behavior, human bone marrow cells containing CTPs were harvested, isolated, and cultured on PDMS substrates comprising smooth surfaces, and channels and posts textures. The proliferation and migration of CTPs on micro-textured PDMS substrates should improve the understanding CTP behavior as well as the capabilities for controlled stimulation. Both of these points are parameters in the engineering of bone scaffolds for the enhancement of bone fracture healing.

Materials and Methods

Experimental Design

Bone marrow derived CTPs were cultured on PDMS substrates comprising smooth (non-patterned) surfaces (SMOOTH), 4 different cylindrical posts micro-textures (POSTS) that were 7-10 μm high and 5, 10, 20, and 40 μm diameter, respectively, and channels textures (CHANNELS) with curved cross-sections that were 11 μm high, 45 μm wide, and separated by 5 μm wide ridges. The primary reason for using PDMS as the substrate material is that it is highly inert, and this permits isolation of the effect of surface micro-textures on cell behavior from other surface effects. The textured PDMS substrates were realized by microfabrication and micromachining technologies. Fresh bone marrow derived cells were plated on the substrates, cultured for 9 days, fixed, analyzed using light, fluorescent, and scanning electron microscopy, and tested for differentiation using in situ staining for alkaline phosphatase activity. Each experiment was repeated three times and the results were compared to those from cells grown on glass tissue culture dishes that served as controls.

Substrate Preparation

Three different kinds of PDMS substrates were manufactured by soft Llithography using similar processs.

POSTS Surfaces

A 1.3 μm-thick layer of photoresist was coated on top of a silicon wafer that had been previously oxidized to grow a 1.5 μm-thick SiO₂ layer. Photolithography was then used to transfer the different texture patterns from a photomask onto the photoresist. Afterwards, the SiO₂ layer was etched using buffered oxide etchant (BOE) to create a patterned mask for selective removal of the silicon substrate by reactive ion etching (RIE). The silicon substrate was etched to a depth of 7-10 μm to create the master for subsequent PDMS molding. The patterned wafer was then coated with 1H,1H,2H,2H—Perfluorodecyltrichlorosilane (Lancaster, Pelham, N.H.) to aid the PDMS release. The liquid PDMS base and curing agent (Sylgard 184) (Dow Corning, Midland, Mich.) components were mixed in a ratio of 10:1, degassed for 7 minutes, and then poured uniformly on top of the patterned master. After additional degassing for 12 minutes, the PDMS on the patterned silicon was cured at 65° C. for 3 hours and at room temperature (˜25° C.) for 21 hours. The cured PDMS cast was released from the master and sectioned into 1 cm×1 cm samples. Four different micro-textures were realized on the PDMS substrates comprising posts that were 7-10 μm high and 5, 10, 20, 40 μm diameter, respectively. The separation between posts on a given substrate was equal to the corresponding post diameter.

CHANNEL Surfaces

An 11 μm-thick layer of photoresist (AZ-9260, AZ Electronic Materials, Somerville, N.J.) was coated on top of a standard 100 mm diameter, (100)-oriented silicon wafer. Photolithography was then performed to transfer straight channel patterns from a photomask to the coated photoresist. The reflow of the photoresist during the final bake step (115° C. for 30 min) of the photolithography process resulted in rounding of the edges of photoresist patterns. This patterned photoresist on the silicon wafer constituted the master for subsequent PDMS molding. The PDMS casting process was the same as that used for the Post textures. The cured PDMS cast was released from the master and sectioned into 1 cm×1 cm samples with curved channels that were nominally 11 μm high and 45 μm wide. The micro-channels were separated by ridges that were flat and 5 μm wide at the top and offset at maximum of 50° from the channel wall.

SMOOTH Surfaces

The SMOOTH PDMS surfaces were created using the same process as the one used for both POSTS and CHANNELS, except that the silicon master was a bare silicon wafer. The cured PDMS substrates were released from the silicon wafers and cut into 1 cm×1 cm samples to realize PDMS substrates with smooth surfaces.

Representative surfaces of POSTS, CHANNELS, and SMOOTH were inspected for defects by scanning electron microscopy (SEM) (JSM-5310, JEOL USA, Peabody, Mass.) before and after sterilization in ethanol as described in the next section.

Cell Culture

Cell cultures were conducted in the different experiments. These experiments included a) quantification of cell number, cell number/colony, colony density, cell alignment, and colony aspect ratio; b) cell staining for actin cytoskeleton, cell nuclei, and focal adhesions, and cell height quantification; c) DNA transfection for live viewing of the actin cytoskeleton; d) time-lapse microscopy for live viewing of cell behavior; and d) SEM observations.

All cell cultures were conducted in the same manner, except those used for DNA transfection. In general, bone marrow aspirates were harvested with informed consent from patients immediately prior to elective orthopaedic procedures. Briefly, a 2 ml sample of bone marrow was aspirated from the anterior iliac crest into 1 ml of saline containing 1000 units of heparin (Vector, Burlingame, Calif.). Human bone marrow is a source of relatively easy isolation of CTPs compared to other tissues. The heparinized marrow sample was suspended into 20 ml of Heparinized Carrier Media (alpha-MEM+2 units/ml Na-heparin; Gibco, Grand Island, N.Y.) and centrifuged at 1500 rpm (400×) for 10 minutes. The buffy coat was removed, resuspended in 20 ml of 0.3% BSA-MEM (Gibco), and the number of nucleated cells was counted. The PDMS substrates and control surfaces were sterilized for 30 minutes with 70% ethanol (Aaper Alcohol and Chemical Co.). Cells were then plated on Day 0 at a seeding concentration of 250,000 cells/ml and cultured for 9 days in α-MEM media (Gibco #11900-073) with 10% Fetal Bovine Serum (Whittaker, Walkersville, Md.) plus Dexamethasone (Sigma-Aldrich #D-1756), which was used to enhance osteoblastic expression. The media was removed and replaced on Days 1, 2, 5, and 7.

Cell Fixation and Staining

Fixation for Cell Quantifications

On Day 9, the cultures were fixed and permeabilized by placing the substrates in acetone:methanol in a 1:1 ratio for 10 minutes. Afterwards, cells were stained with 6-diamidino-2-phenylindole dihydrochloride hydrate (DAPI) (Vector), a nuclear fluorescent stain, for 6 minutes at 25° C. and subsequently washed three times with phosphate buffer. After imaging nuclei using DAPI staining, cells were again stained in situ for alkaline phosphatase (ALP), a marker for osteoblastic differentiation, using 0.9 mM Napthol AS-MX phosphate and 1.8 mM Fast Red-TR Salt for 30 min at 37° C. This staining forms an insoluble Napthol-Fast Red complex that precipitates in regions where cells express ALP activity. The precipitate was observed after autofluorescence.

Fixation for Actin and Focal Adhesion Staining

Other SMOOTH, POSTS, AND CHANNELS surfaces were used for staining actin and phosphotyrosine, to observe the cell actin cytoskeleton and focal adhesions, respectively. These cells were fixed with 2% paraformaldehyde (Electron Microscopy Sciences, Washington, Pa.) in PBS for 10 min, rinsed three times with PBS, permeabilized for 10 min with 0.2% Triton X-100® (Lab Chem Inc., Pittsburgh, Pa.) in PBS, and rinsed again three times with PBS prior to both phosphotyrosine and actin immunostaining. Specimens to be immunostained for phosphotyrosine were incubated with a primary antibody AB 4G10 (Upstate Biotechnology, Lake Placid, N.Y.) at 1:500 in PBS for 2 hr at 25° C., rinsed with PBS, followed by a secondary antibody Alexa 546 (Molecular Probes, Eugene, Oreg.) at 1:20 in PBS for 1 hr at 25° C. After exposure to the secondary antibody, the specimens were rinsed three times with PBS, and then mounted with DAPI-containing Vectashield as stated above. Specimens stained for filamentous actin were incubated with Rhodamine Phalloidin (Sigma, St. Louis, Mo.) at 1:50 in PBS for 45 minutes at 25° C., rinsed three times with PBS, and then mounted with DAPI-containing Vectashield as stated above.

Staining Using DNA Transfection

Another group of cells cultured were transfected with a bacterial plasmid tagged with a Green Florescent Protein (GPR). Cells cultured for 5 days on the 10 μm POSTS and SMOOTH surfaces were incubated for 5 hr with pEGFP-actin (1.25 μg/substrate, human cytoplasmic β-actin with GFP on the C terminus, Clonetech, Palo Alto, Calif.). The cells were washed with DME/Ham's F-12 medium containing 5% serum and placed back into the incubator to culture for 3 more days. The live cells were then observed under a fluorescent microscope and the transfected cells analyzed using time-lapse microscopy.

Fixation for SEM Observations

SEM was used to observe cell morphology. In order to assess the possibility of cell damage due to cell fixation, two different procedures were used to prepare the cells for SEM observation. On Day 9, the media was removed and the plated substrates were immersed in a solution containing 2% glutaraldehyde (Electron Microscopy Sciences, Fort Washington, Pa.), 3% sucrose (Sigma-Aldrich Co., Irvine, UNITED KINGDOM), and 0.1 M phosphate buffer (Baxter, Deerfield, Ill.) at 4° C. and 7.4 pH. After 1 hour, substrates were rinsed twice with the phosphate buffer for 30 minutes at 4° C. and washed with deionized (DI) water for 5 minutes. Dehydration was achieved by placing the plated substrates in 50% ethanol (in DI) for 15 minutes and replacing it every 15 minutes while increasing the concentration of ethanol to 60, 70, 80, 90, and finally 100% (Aaper Alcohol and Chemical Co.). The liquid ethanol was removed using critical point drying. The second fixation protocol followed the same steps, except that dehydrated cells were immersed for 5 minutes in hexamethyldisilazane (HMDS) instead of using critical point drying.

Cell Culture Analysis

Cell Quantification

A phase contrast microscope (Olympus CK2) (Olympus Optical Co., JAPAN) was used for daily observation of the cells. Cells were fixed on Day 9, stained with DAPI and viewed under a fluorescent microscope (Olympus BX50F) (Olympus Optical Co.) to study different cell parameters: cell attachment, by counting the number of colonies that are formed; cell proliferation, by counting the number of cells within a single colony; cell migration, by studying the distribution of cells within the colony; and cell differentiation, by assay of markers of the osteoblastic phenotype. To verify this quantification technique, additional cells were cultured and the cell number quantified by photographing with the fluorescent microscope (Olympus BX50F, Olympus Optical Co.) five randomly chosen fields of vision at 4×, and counting the number of cells in these fields of vision.

In addition to cell number/colony and cell number, the following colony parameters were also quantified: colony area, colony density, length of longest axis of the colony (MAXL), and length of shortest axis of the colony (MINL). A colony was defined as a cluster of 8 or more cells. ¹⁸ Colony area, MAXL, MINL, and the resulting colony aspect ratio (AR=MAXL/MINL) were quantified using the computer software Image-Pro Plus (Media Cybernetics, Inc., Silver Spring, Md.). In order to account for random variations between and within experiments, statistical significance was defined at the 95% confidence interval using Analysis of Variance (ANOVA) test performed in Microsoft Excel (Microsoft Corp., Redmond, Wash.). Cell alignment was quantified by photographing five randomly chosen fields of vision of CTPs on CHANNELS textures, and measuring the angle between the longest axis of the cell and the channel axis. The morphology of cells cultured on the PDMS substrates and control surfaces was also examined using SEM, and the actin cytoskeleton and focal adhesions were observed using a confocal microscope (Leica TCS-SP Laser Scanning Confocal Microscope, Heidelberg, GmBH, Germany). Finally, cells were stained in situ for ALP and viewed using a fluorescent microscope to verify differentiation into osteoblastic phenotype.

Analysis of Other Experiments

SEM observations were used to qualitatively describe the morphology of the cells growing on the different substrates. The actin cytoskeleton and focal adhesions on POSTS, CHANNELS, and SMOOTH surfaces were observed with the confocal microscope on randomly chosen cells. Two types of heights were quantified on cells grown on POSTS; the height from the lowest observed actin to the bottom part of the cell nuclei, and the height from the lowest actin to the top part of the cell nuclei. The results show the mean heights from 20-30 quantified cells on 10, 20, 40 μm POSTS and SMOOTH surfaces.

Time-lapse microscopy was used to observe the cells transfected with DNA on the 10 μm POSTS under a Leica DM IRB fluorescent microscope (Leica, Heidelberg, GmBH, Germany), and the cells cultured on 10 μm POSTS, CHANNELS, and SMOOTH surfaces using the same microscope in a phase contrast mode. This microscope was equipped with an incubator that controlled temperature and CO₂ for live viewing of the cells.

Results and Discussion

SEM examinations revealed that the PDMS substrates did not exhibit significant geometrical variations with respect to the photoresist/silicon master. Furthermore, a comparison of the PDMS substrates before and after ethanol sterilization did not reveal any apparent pattern degradation. The effect of the SEM fixation procedure on the PDMS substrates was minimal; only slight markings were observed on PDMS surfaces immersed in HMDS. Qualitative SEM examinations revealed that morphologies of cells on the various PDMS substrates were similar for both critical point drying and HMDS-based fixation procedures.

Cell Morphology and Distribution

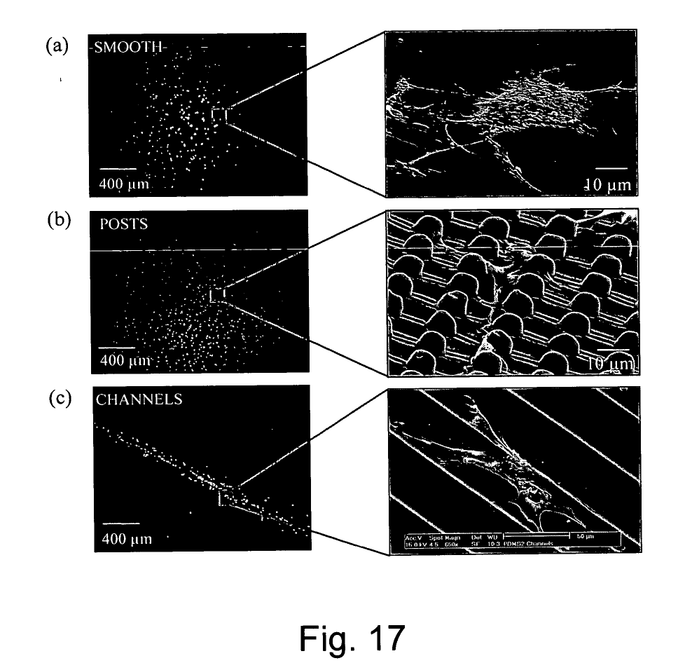

CTPs attached, proliferated, migrated, and differentiated on all PDMS substrates and control surfaces. There were clear differences at the cell and colony level between cells grown on all three PDMS substrates (FIG. 17a-c).

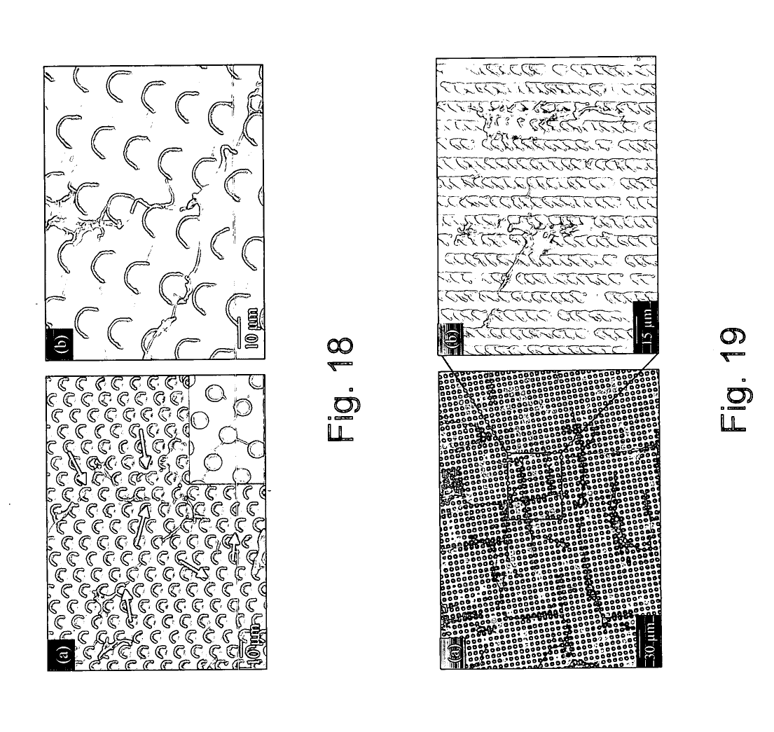

Before spreading, the cells exhibited a circular shape of approximately 10-12 μm diameter, and when spread, the cell morphology varied with the culturing substrate. On the SMOOTH PDMS and control surfaces, cell bodies adopted a random flattened shape, ranging from 40-100 μm-diameter and exhibited average process lengths up to 80 μm (FIG. 17a). The cells on POSTS tended to attach next to the posts and spread between them, exhibited highly contoured morphology, and directed their long processes (up to 300 μm) towards posts and other cells (FIG. 18).

Qualitatively, cells cultured on POSTS with smaller-diameter posts were smaller and exhibited narrower processes than those grown on larger-diameter posts with correspondingly greater distances between posts. Similar observations have been previously described by Schmidt and von Recum. The narrowest processes were observed on cells cultured on the 5 μm POSTS, in which individual cells tended to grow between and along the array of posts (FIG. 18). Similar orthogonal alignment was also observed for most of the cells cultured on the 10 and 20 μm POSTS. An equivalent channel-like texture is also provided by the short separation between posts on the 5, 10, and 20 μm POSTS, which would explain the corresponding cell alignment.

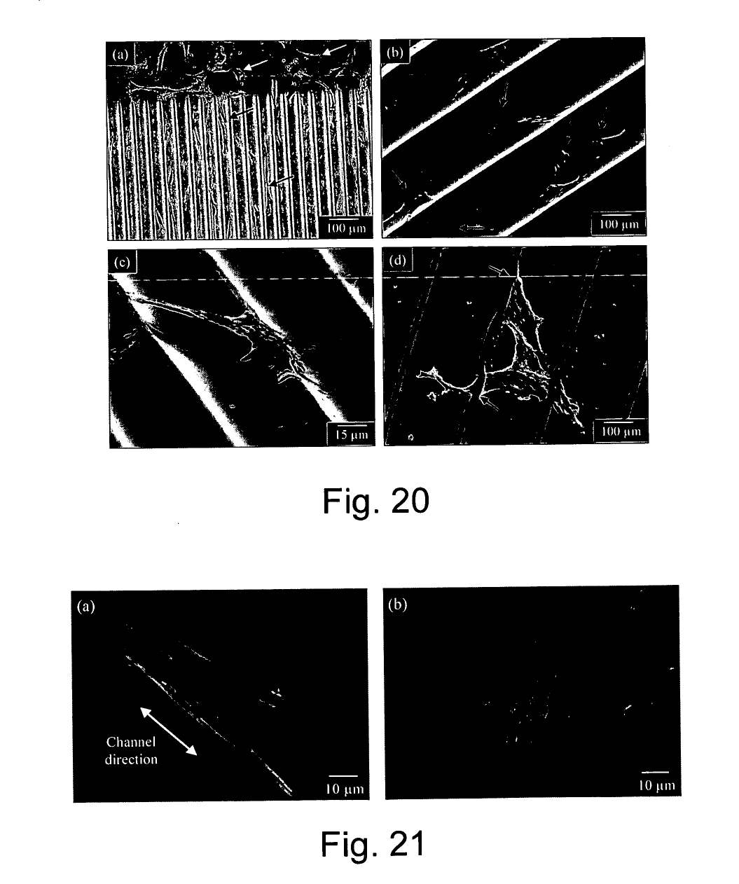

In CHANNELS, cells tended to attach and spread mostly within and along the channels with a mean alignment of 14.44°. Although the process lengths were comparable to those on cells grown on the smooth surfaces, cell bodies were narrower and were oriented along the channel axis. When cells encountered a region where channels and smooth met, cells on channels aligned, while cells on smooth kept their random orientation (FIG. 19). The majority of CTPs on CHANNELS were located within the channels and aligned to the channel axis with the major processes generally directed towards those of other cells (FIG. 20b). However, a few cells straddled the ridges between channels (FIG. 20c). SEM and phase contrast microscope images revealed distortions on ridges at points of attachment by cell processes (FIG. 20d), which suggests strong adhesion that is likely mediated by either adsorbed serum proteins from the medium to the surface, or by cell attachment factors expressed by CTPs on the PDMS surfaces.

Cell shape has been implicated to participate in the regulation of cell differentiation. However, our qualitative observations revealed similar ALP staining on all the surfaces, which suggested that surface texture did not affect their differentiation into osteoblastic phenotype.

Actin Cytoskeleton, Focal Adhesions, and Cell Height

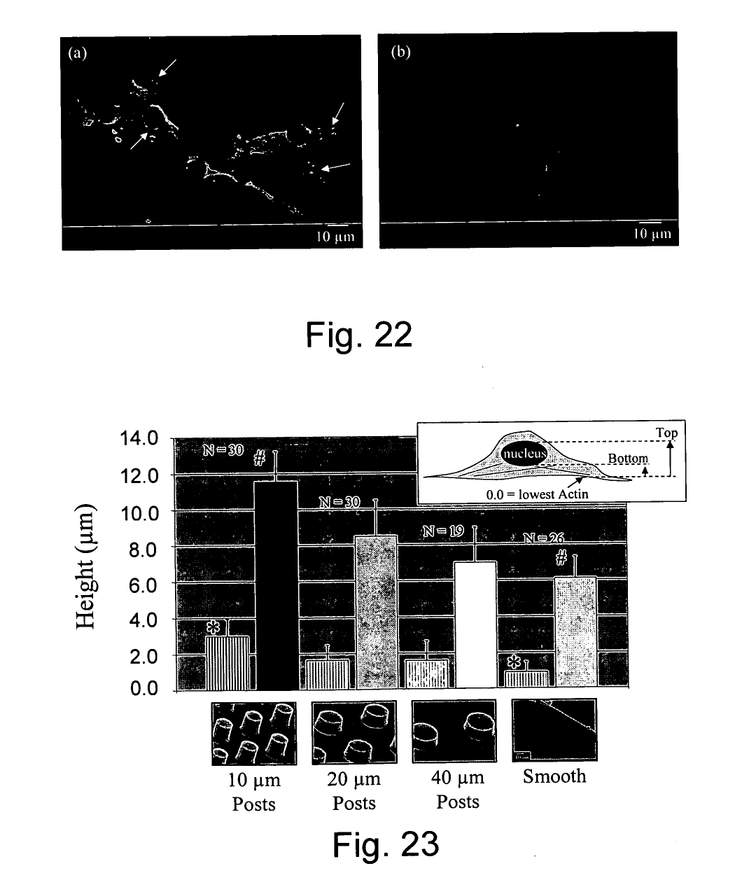

As expected, the actin cytoskeleton from cells on CHANNELS was aligned along the direction of the channels, compared to a more random orientation on cells growing on SMOOTH surfaces (FIG. 21), which is consistent with previous reports.⁵⁰ On POSTS, the actin microfilaments were closer together (perhaps due to a narrower body as observed from the SEM), and were directed towards the posts; while focal adhesions were seen on the bottom and around the post textures (FIG. 22).

SEM observations suggesting a higher contoured cell morphology on smaller size POSTS were confirmed by the cell height quantification. The height of the cell nuclei was quantified from the lowest observed actin microfilaments on the different surfaces. From these measurements, it was observed that the position of the cell nuclei above the lowest actin increased in height as the cells grew from the most spacious SMOOTH surface (0.9 μm) to the narrowest quantified 10 μm POSTS (3.0 μm) (FIG. 23). In addition, the height of the cell nuclei also increased from 6.1 μm on SMOOTH to 11.5 μm on the 10 μm POSTS. This data was obtained by subtracting the heights point of the nuclei minus the lowest point of the nuclei on the different surfaces.

Time-Lapse Microscopy

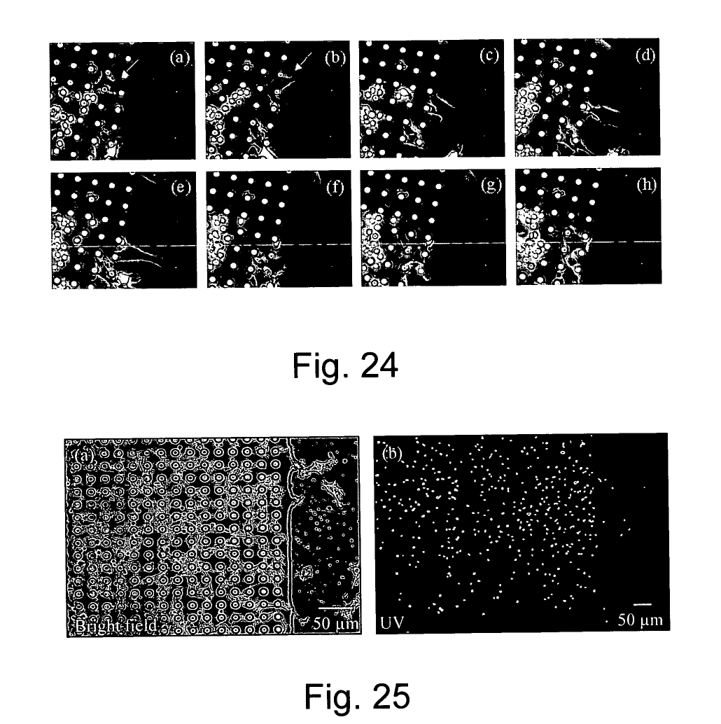

Time-lapse microscopy using phase contrast mode allowed visualization of cell proliferation and migration on POSTS, CHANNELS, and SMOOTH. Cell behavior was different on cells growing on all three surfaces. In POSTS, cells migrated within the posts acquiring thinner shapes and higher contours than cells on SMOOTH, and appeared to preferentially grab the posts as they moved within them. Cells on SMOOTH appeared to anchor in random locations on the surface as they migrated. At the points of contact between POSTS and SMOOTH, cells were observed to grab on the posts and either stay within the posts or migrate towards the posts (FIG. 24). This behavior can be seen in FIG. 25 where a fixed cell colony, stained with DAPI, clearly shows the preference of cells to stay and grow on the POSTS compared to the SMOOTH.

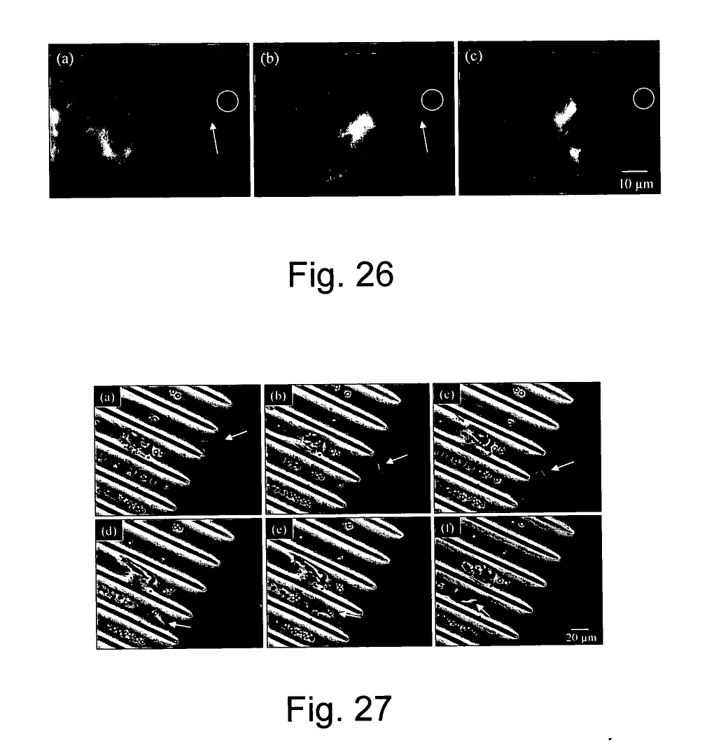

Observations on the transfected cells confirmed that cells growing on POSTS directed the actin microfilaments towards the posts (FIG. 26). It was noticed that cells attached to the posts by extending their cytoskeleton and surrounding (grabbing) the posts as the celled migrated between them.

In CHANNELS, cells seemed to align along the direction of the channels, while positioning their body within the channel space and extending their processes towards the ridges (elevated areas) of the channels. In addition, cells seemed to have a meandering migration pattern within the two ridges located on each side of the channel space. At the points of contact between CHANNELS and SMOOTH, cell bodies seemed to narrow as they entered the channels and aligned within them (FIG. 27). Unlike cells on POSTS, there was no evidence that cells preferred to grow within the CHANNELS compared to SMOOTH.

Cell and Colony Quantification

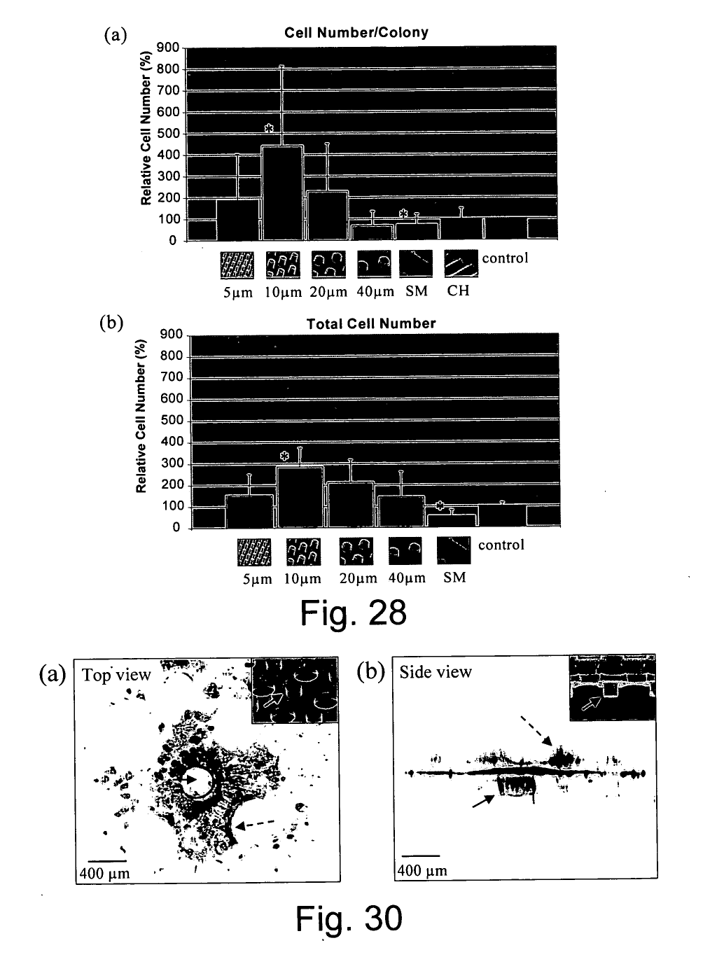

CTPs attached, proliferated, and differentiated on all PDMS substrates and control surfaces. FIG. 28, present results from the cell culture analysis for different cell and colony parameters on POSTS, CHANNELS, SMOOTH and control surfaces. It is known that humans differ significantly from one another with respect to the cellularity of bone marrow and the prevalence of osteoblastic progenitor cells, and therefore, the cell counts on the PDMS substrates are normalized to the control surfaces within the particular experiment (same donor). The mean and standard deviation parameters were derived from three different experiments. The AR values were computed from the corresponding MAXL and MINL (not shown) values.

Cell Number/Colony and Cell Number

On Day 9, SMOOTH exhibited a mean number of cells per colony of 71% those on the control surfaces (100%). The difference in cell number/colony between Smooth and control might be partially explained by the difference in critical surface tension between the PDMS (24 dynes/cm, hydrophobic) and glass (170 dynes/cm, hydrophilic). In contrast, all POSTS had increased numbers of cells per colony compared to both the SMOOTH and control surfaces. The maximum number of cells/colony was observed on the 10 μm POSTS, which exhibited around four times (442%) the number of those on the control and a statistically significant difference compared to the SMOOTH surface (p<0.05). Cell number/colony on the 20 and 5 μm POSTS nearly doubled (228% and 188%) that of the control surface, while 40 μm POSTS exhibited the lowest cell number/colony (67%) and CHANNELS (98%) presented similar cell number/colony than the control surface. Likewise, cell number quantified from the five random fields of view followed the same trend, where POSTS had increased numbers of cells compared to SMOOTH and control surfaces. As in the previous experiments, the maximum number of cells was observed on the 10 μm POSTS, which exhibited nearly three times (286%) the number of those on the control and a statistically significant difference compared to the SMOOTH (p<0.05). Cell number on 20 μm PDMS POSTS textures was almost double (213%) that of the control surface, while the 5 and 40 μm POSTS textures exhibited cell numbers that were 153% and 144% that of the control surface, respectively. In general, this experiment exhibited the same trend as the previous one.

These results contrast those reported where the tissue culture dishes exhibited significantly higher fibroblast and macrophage proliferation compared to corresponding PDMS post textures. Although the reasons for the discrepancy between our observations and the previous reports are not clear, it is possible that the difference in cell types and corresponding surface affinities, along with variations in topography and biochemical stimuli, might be contributing factors.

Colony Density

The size of cell colonies on the different substrates was different, and correlated with the cell number/colony results. Areas of cell colonies on SMOOTH (0.96 mm²) and control surfaces (1.05 mm²) were comparable, but significantly smaller than the areas of the cell colonies on POSTS, especially compared to the 10 μm POSTS (6.57 mm²). Nonetheless, CHANNELS exhibited the smaller size colonies with 0.64 mm². Furthermore, an examination of cell density within colonies (cell number per unit area) reveals that colonies on CHANNELS (229% denser than control) tended to be denser than colonies on POSTS (up to 140% on 10 μm POSTS), and were significantly denser than those on SMOOTH (104%). These observations collectively suggest that the rate of migration of cells within colonies on CHANNELS was significantly lower than on SMOOTH and control surfaces. It is believed that this fact is not due to fusion of multiple colonies since the number of colonies was similar on all POSTS, CHANNELS, and SMOOTH substrates.

Colony Aspect Ratio

Colonies on POSTS, SMOOTH, and control surfaces were similar and exhibited arbitrary shapes without any preferred orientation (AR˜1). In contrast, colonies on CHANNELS were highly directional and appeared elongated along the direction of the channel axes, with a mean MAXL of 3252 μm. POSTS exhibited lower MAXL values (1744 μm on 40 μm POSTS to 3086 μm on 10 μm POSTS), while SMOOTH presented significantly lower mean MAXL of 1265 μm. The directionality of the CTP migration is reflected by the higher AR values of colonies on the CHANNELS with AR of 13.72 compared to 1.57 on SMOOTH, 1.51 on control, and 1.26 and 1.51 on 20 μm and 40 μm POSTS, respectively.

The drastic increase in colony AR and MAXL for the CTPs cultured on PDMS CHANNELS textures are important parameters that should be considered when designing not only bone, but a variety of tissue engineering constructs that use CTPs. These effects may be particularly applicable when cells need to be localized in specific areas or when cell migration needs to be directed.

Conclusion

Microfabrication and micromachining technologies enable reproducible patterning of precise surface micro-textures, which can be exploited to investigate cellular response to surface topography. The information obtained from such investigations is particularly crucial to the successful development of three-dimensional scaffolds that can optimize variables that influence cellular behavior, such as surface topography.

These results demonstrate that culturing of CTPs on micro-textures can modify cell morphology, attachment, migration, and proliferation, and have the potential to enhance or diminish specific cell behaviors. Colonies of cells on 10 μm POSTS exhibited higher cell number than any other post size texture, and a significant increase in cell number (442%) compared to colonies on SMOOTH (71%). In addition, CHANNELS significantly enhanced cell density and colony directionality. The results demonstrate a significant response of CTPs to topography, and suggest a practical role for textured materials in modifying CTP behavior.

Most investigations into the effects of micro-textured surfaces comprising micro-textures for in vitro cell studies have concentrated on individual cell morphology and not colony characteristics. In addition to knowledge of the effects of topography on individual cells, the successful engineering of functional tissues will require understanding how cells behave with their neighbors. Therefore, in vitro colony investigations can be clinically relevant in designing and predicting local tissue reactions to bone implants. The present study is unique not only in combining CTPs with microfabricated surfaces, but also in quantifying both cell and colony behavior as a consequence of micro-topographies. Knowledge of CTP response to surface stimuli could lead to the incorporation of specific micro-textures into surfaces of bone implants to achieve surface-textured 3D scaffold materials for bone grafting or augmenting of fracture healing. From the cell number/colony quantification on POSTS, it is demonstrated how small differences in the surface micro-textures can have either small or dramatic effects on cell behavior. Therefore, there is great importance in using a scaffold fabrication technique such as microfabrication, to offer the development of precise surface micro-textures with micrometer precision that can selectively stimulate specifically cell behaviors.

### Example 2

A 3D Scaffold with Precise Micro-Architecture and Surface Micro-Textures for Bone Tissue Engineering

Microfabrication related techniques such as soft lithography offer precision and reproducibility to develop surface micro-textures that can selectively stimulate cell behavior. However, the use of these techniques to fabricate 3D micro-architectures with surfaces that include desirable micro-textures is hindered by inherent 2D fabrication characteristics of traditional photolithography-based processes. Consequently, the development of a novel 3D manufacturing technique that combines microfabrication and soft lithography to construct 3D scaffolds with precise micro-architecture and surface micro-textures that are designed to selectively stimulate bone cells and tissue. In addition, the behavior of human CTPs on these scaffolds is investigated. This strategy may allow the development of 3D scaffolds that use their geometrical features to maximize osteoconductive stimuli of cells and subsequent bone formation.

Materials and Methods

3d Scaffold Fabrication

The 3D scaffolds were produced through an innovative approach that used microfabrication and soft lithographic techniques and consisted of the dual-sided molding and stacking of Polydimethylsiloxane (PDMS) layers. PDMS was chosen because it is a well-studied flexible material that can be micro-textured, is highly biocompatible and inert, tough, simple to manufacture and manipulate, and well suited for biological applications such as catheters, drainage tubing, insulation for pace makers, membrane oxygenators, and ear and nose implants.

The unique feature of the current scaffold fabrication technique is that it allows the development of pre-defined and precise micro-architectures and surface micro-textures. Most bone ingrowth has been observed in pore diameters between 300-500 μm. Therefore, we report the fabrication of scaffolds that have 300 μm diameter meandering vertical pores and 200 μm×400 μm horizontal pores that were designed to enhance cell penetration, extracellular matrix production, and neovascularization formation. In addition, every horizontal surface within the scaffold comprised 10 μm diameter and 10 μm high posts that were chosen because they have been shown to enhance CTP growth in vitro.

The fabrication process consisted of three basic steps: a) mold fabrication, b) dual-sided molding, and c) stacking of PDMS layers.

Mold Fabrication

First, a multilevel SU-8 photoresist (MicroChem Corp., Newton, Mass.) process was developed to produce SU-8 molds incorporating holes and posts of various dimensions. Three layers of SU-8 were processed on a standard 100 mm-diameter, 500 μm thick, n-type (100)-oriented silicon wafer as follows. First, a 200 μm thick film of SU-8 2100 was spin coated, soft baked in a C-005 convection oven (Lindberg/Blue M, Asheville, N.C.) (95° C., 55 minutes (min)), exposed (365 nm, 375 mJ/cm²), and post exposure baked (95° C., 25 min). A 10 μm thick film of SU-8 2010 was then spin coated, soft baked (95° C., 5 min), exposed (100 mJ/cm²), and post exposure baked (95° C., 5 min). Next, a 1100 μm thick film of SU-8 2100 was spin coated, soft baked, exposed, and post exposure baked using the same process parameters as the first film. Finally, all three SU-8 layers were simultaneously developed in SU-8 Developer (MicroChem Corp.) (25° C., 50 min) using agitation, to realize a multilevel SU-8 mold with 200, 10, and 100 μm-high features.

A second mold was fabricated out of PDMS by spin coating a 10 μm thick film of SU-8 2010 on a standard 100 mm-diameter, 500 μm thick, n-type (100)-oriented silicon wafer, soft baked, exposed, and post exposure baked using the same protocol as the one used for the previous mold. The SU-8 was then developed in SU-8 Developer with agitation for 12 minutes to realize a surface with 10 μm diameter post micro-textures. Finally, this SU-8 micro-textured surface was then used to cast and realize the PDMS mold comprising 10 μm diameter and 10 μm deep holes. This second mold was made of PDMS because its transparency and flexibility facilitate alignment and subsequent separation of the two molds during dual-sided molding of the final PDMS layer.

Dual-Sided Molding

The PDMS and triple-layer-patterned SU-8 molds were coated with 1H,1H,2H,2H-Perfluorodecyltrichlorosilane (Lancaster, Pelham, N.H.) to aid the release of the molded PDMS layer. PDMS was mixed as previously described, poured on top of both molds, distributed to cover all the patterned areas of the molds, and degassed for 15 min. Then, both molds (containing uncured PDMS) were placed on a custom mechanical jig (FIG. 8.2, chapter VII), which allows horizontal, vertical, and rotational motion control for alignment of the molds within about ±10 μm.

The two molds were then aligned and brought to contact (with the patterned sides facing each other) while squeezing the uncured PDMS. The jig was then placed inside of an oven at 75° C. for 2 hours to cure the PDMS. After curing, the two molds were removed from the jig, allowed to cool to room temperature, and immersed in methanol to remove (or separate) both molds from the squeezed PDMS. The patterned PDMS layer was then cut with a blade into ˜1.5 cm×1.5 cm specimens.

Stacking of PDMS Layers

The patterned PDMS layer specimens were stacked using uncured PDMS as adhesive. PDMS was prepared as explained above, poured to cover about ⅔ of a smooth 100 mm diameter, 500 μm thick, n-type (100)-oriented silicon wafer, and spin coated using a 400 Lite spinner (Laurell Technologies, North Wales, Pa.) at 4000 revolutions per minute (rpm) to achieve a ˜10 μm thick layer. The cured and patterned specimens were stamped on top of the uncured PDMS, so that the tips of the 200 μm columns were wetted with uncured PDMS. Then, the layers were handled with tweezers and stacked one on top of the other after being aligned using a light microscope. The aligned and stacked PDMS layers were then baked at 95° C. for 30 minutes to cure the PDMS (adhesive) and bond the PDMS layers. Lastly, the 3D structures were cut using either a 1 cm diameter circular die to realize scaffolds for cell experiments (5 PDMS layers), or a blade to obtain samples for scanning electron microscope (SEM) observations (5-12 PDMS layers).

CTPSs on 3d Scaffolds

The 3D scaffolds with precise micro-architecture and surface micro-textures (3D PDMS Texture) were then used as substrates for CTP culture in vitro to determine their effect on CTP behavior. In order to compare the effect of these scaffolds on CTP growth, another set of 3D scaffolds were fabricated with the same steps of mold fabrication, dual-sided molding, and stacking of PDMS layers, with the exception of the 10 μm micro-textures. This process realized 3D scaffolds with exactly the same micro-architecture but with smooth surfaces instead of micro-textured surfaces (3D PDMS Smooth).

Cell Culture

CTP Preparation

Bone marrow aspirates were harvested from patients immediately prior to elective orthopaedic procedures. Briefly, a 2 ml sample of bone marrow was aspirated from the anterior iliac crest into 1 ml of saline containing 1000 units of heparin (Vector, Burlingame, Calif.). The heparinized marrow sample was suspended into 20 ml of Heparinized Carrier Media (alpha-MEM+2 units/ml Na-heparin; Gibco, Grand Island, N.Y.) and centrifuged at 1500 rpm (400×) for 10 min. The buffy coat was removed and resuspended in 20 ml of 0.3% BSA-MEM (Gibco) for subsequent inoculation of the cells on the scaffolds.

3D Scaffold Set Up and Cell Inoculation

The 3D PDMS Texture and 3D PDMS Smooth scaffolds were sterilized for 30 min with 70% ethanol (Aaper Alcohol and Chemical Co.), followed by rinsing three times with phosphate buffered saline (PBS) (Mediatech, Inc. Herndon, Va.). Each scaffold was then placed inside of a 10 ml syringe for loading of the cells. Prior to cell inoculation, the cells were diluted in 2.5 ml of α-MEM media (Gibco #11900-073) with 10% Fetal Bovine Serum (Whittaker, Walkersville, Md.) plus Dexamethasone (Sigma-Aldrich #D-1756), counted using a hemacytometer, and loaded in a 10 ml syringe. The syringe was placed on a NE-500 syringe pump (New Era Pump Systems, Wantagh, N.Y.), which was operated to pass the 2.5 ml of media containing cells at 1.5 ml/min trough the scaffolds. The effluent media and cells were collected and counted to determine the cell loading efficiency into each scaffold.

A separate cell culture was set up in order to determine if cells were able to migrate in a vertical upward direction within the scaffolds. In this experiment, cells from the same population as those inoculated into the scaffolds were cultured in a glass tissue culture dish (Lab-Tek, Nalge Nunc Int., Naperville, Ill.). On Day 9, a 3D PDMS Texture scaffold was placed on top of the live cells with the 200 μm columns facing down, so that the tips of these columns were in contact with the cells in culture. This set up was then cultured for another 4 days, after which the scaffold was removed to investigate whether cells were migrating up from the tissue culture dish into the scaffold.

Cell Culture and Analysis

The loaded scaffolds were placed on a Lab-Tek tissue culture dish (Nalge Nunc Int.) and inside of an incubator, where cells were cultured for 9 days in A-MEM media (Gibco #11900-073) with 10% Fetal Bovine Serum (Whittaker, Walkersville, Md.) plus Dexamethasone (Sigma-Aldrich #D-1756), which was used to enhance osteoblastic expression (media changed on days 1, 3, 5, and 7). After fixation of the cells on Day 9, they were stained with 6-diamidino-2-phenylindole dihydrochloride hydrate (DAPI) (Vector), a nuclear fluorescent stain. A fluorescent microscope (Olympus BX50F) (Olympus Optical Co.) and confocal microscope (Leica TCS-SP Laser Scanning Confocal Microscope (Hedelberg, GmBH, Germany) were used to determine the colonies and count the cell number per colony. The cells were counted by taking advantage of the transparency of the PDMS layers and the orthogonality of the micro-architecture (horizontal and vertical walls), which facilitated the quantification of the cells by focusing the microscopes on different depths of the colonies and counting the cells on the different levels. Cell growth was characterized by quantifying the cell number per colony in order to investigate proliferation characteristics on the different scaffold surfaces, and avoid potential differences in cell loading efficiency. Cell experiments were carried out in triplicate with cells from three different human donors. In order to account for random variations between and within experiments (due to donor variability), statistical significance was defined at the 95% confidence interval using analysis of variance (ANOVA) test performed in SigmaStat (SPSS Inc., Chicago, Ill.).

Other cell-loaded scaffolds were used for staining of the cell nuclei and actin cytoskeleton, to observe cell distribution within the scaffolds using the fluorescent and confocal microscopes. These cells were fixed with 2% paraformaldehyde (Electron Microscopy Sciences, Washington, Pa.) in PBS for 10 min, permeabilized for 10 min with 0.2% Triton X-100 (Lab Chem Inc., Pittsburgh, Pa.) in PBS, and rinsed three times with PBS. Cells were then stained with Rhodamine Phalloidin (Sigma, St. Louis, Mo.) at 1:50 in PBS for 45 min at 25° C. for the actin cytoskeleton, rinsed three times with PBS, and stained with DAPI for the nuclei.

Results and Discussion

Scaffold Fabrication

SEM examinations revealed that the three-level SU-8 molds had the desired geometrical dimensions of 200 μm diameter and 200 μm deep holes, 300 μm diameter and 100 μm high columns, and 10 μm diameter and 10 μm deep holes. This mold fabrication technique is advantageous because it combines the precision of microfabrication with the complexity and three-dimensionality of multiple SU-8 layering, while using a single developing step. This approach allowed the fabrication of up to six SU-8 levels while reducing processing time and avoiding processing complications of coating over patterned surfaces, which would hinder exposure uniformity, feature resolution, and alignment during the dual-sided molding of the PDMS.

SEM observations also revealed PDMS layers ranging between 80-120 μm thick, with 300 μm diameter through holes (formed at the points where both molds were in contact), 10 μm posts on one side of the layer (from the PDMS mold), and 200 μm columns along with 10 μm posts on the other side (from the triple-layer-patterned SU-8 mold). The custom mechanical jig significantly enhanced the alignment of the two molds used for dual-sided molding (“sandwiching”) of the PDMS. In this step, special care was given to ensuring uniform contact between the two molds to avoid blocked through holes and non-uniform thickness of the PDMS layers. Nonetheless, the resulting PDMS layers had a ±20 μm variation from the desired 100 μm thick PDMS layer.

Alignment of the PDMS layers was facilitated by the transparency of the PDMS, where the 300 μm diameter through holes and 200 μm diameter columns served as alignment marks during the stacking step with a resulting alignment accuracy of +50 μm between the stacked PDMS layers. Stamping uncured PDMS onto the 200 μm columns as adhesive was a convenient way to attach the different layers. However, care had to be taken to assure a uniform stamping/wetting of the tips of these columns, as well as subsequent contact between PDMS layers during stacking. If the 200 μm diameter columns of the PDMS layers were brought into contact unevenly with the uncured PDMS (adhesive), over-wetting of some columns would result, which, in turn, could lead to destruction of nearby micro-textures.

A 5 layer 3D PDMS Texture scaffold was formed that exhibits 66% porosity by volume with 300 μm diameter meandering vertical pores, 200 μm×400 μm horizontal pores, and 71% of the surfaces within the scaffold covered with 10 μm diameter and 10 μm high posts. The height (three-dimensionality) of the resulting 3D scaffolds can be increased by simply stacking more PDMS layers.

CTPS within the 3D Scaffolds

The effect of the 10 μm high and 10 μm diameter posts on CTP behavior was investigated by culturing CTPs on the 3D PDMS Texture scaffolds, and comparing them with CTP cultures on the 3D PDMS Smooth scaffolds. These scaffolds had the same pore size and geometry as the 3D PDMS Texture scaffolds, but smooth surfaces instead of surface micro-textures.

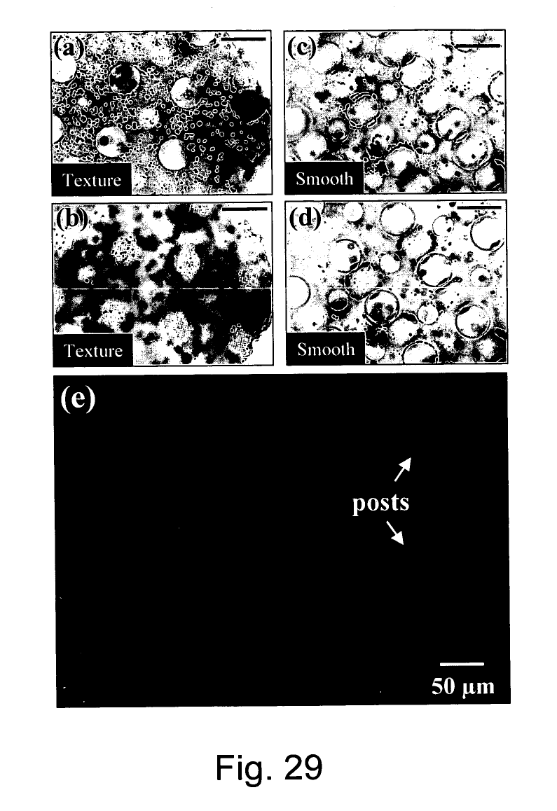

Cells attached, migrated, and proliferated in all three dimensions on the different features of the micro-architecture within the scaffolds. CTP colonies were visible on different levels of both 3D PDMS Texture and 3D PDMS Smooth scaffolds, with cells growing from 1-4 PDMS layers deep per colony. Cells on the different colonies were visible growing on the top and bottom of each PDMS layer, on the walls of the 300 μm pores, and on the 200 μm columns linking the different PDMS layers (FIG. 29). In addition, confocal microscope observations exhibited a clear actin cytoskeleton that seemed to grow within and around the posts (FIG. 29e). No colonies were observed to expand the 5 layers of the scaffolds. These observations suggest that in the vertical direction, cells proliferated and migrated to cover distances of up to about 1.2 mm long, while in the horizontal direction some colonies reached diameters of about 3.0 mm long. The increased distance covered by cells in the horizontal direction may be a result of cells preferring to grow within the post micro-textures present on all horizontal surfaces, compared to smooth areas of the vertical surfaces. Nevertheless, cells were able to migrate vertically, including upward as was revealed by cells growing from the tissue culture dish into the 3D PDMS Texture scaffold (FIG. 30).

On the first two experiments, cell-loading efficiency was similar on both 3D PDMS Texture (47%, 80%) and 3D PDMS Smooth (60%, 74%) scaffolds. On the third experiment, 3D PDMS Texture (4%) had considerably less number of cells attaching on it compare to cells on 3D PDMS Smooth (30%). This lower cell loading efficiency on 3D PDMS Texture may have resulted from shifting of the scaffold during cell loading, or from air bubbles present within the scaffold that blocked passing and therefore loading of the cells. Table 1 shows that colony number on both scaffolds was related to the number of cells loaded on each scaffold, with similar colony number for all scaffolds, and lower number of colonies on 3D PDMS Texture for the third experiment.

TABLE 1

| | Scaffold | Cell Loading (%) | Colony Number |
|---|---|---|---|
| Exp 1 | Texture | 47 | 9 |
| | Smooth | 60 | 9 |
| Exp 2 | Texture | 80 | 5 |
| | Smooth | 74 | 4 |
| Exp 3 | Texture | 4 | 8 |
| | Smooth | 30 | 14 |

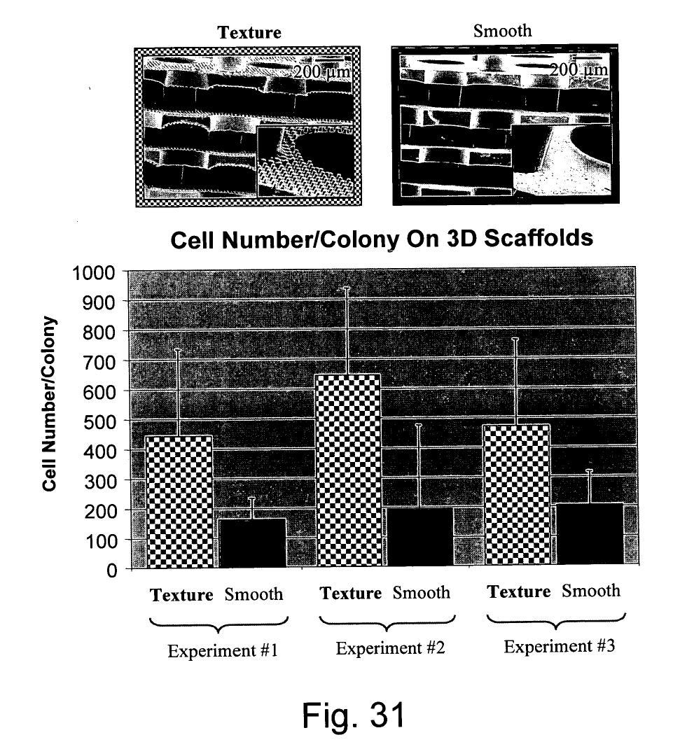

On all three experiments, 3D PDMS Texture scaffolds consistently presented more cells per colony (443, 645, 470) than 3D PDMS Smooth (162, 194, 203) (FIG. 29 and FIG. 31). The mean cell number per colony was 499 for 3D PDMS Texture compare to 188 cells per colony on 3D PDMS Smooth, which represent a statistically significant difference (p<0.05) between the two types of 3D PDMS scaffolds. These results are consistent with those of previous experiments on CTPs cultured on 2D surfaces comprising 10 μm high and 10 μm diameter post micro-textures. In addition, CTP morphology, migration, and proliferation observed on the post micro-textures and smooth surfaces within the 3D scaffolds were similar to those CTP behaviors observed earlier on similar posts and smooth regions of two-dimensional (2D) surfaces.

Conclusion

The use of a 3D scaffold that supplies CTPs with osteoconductive conditions may provide an efficient bone graft alternative that avoids the complications related to the use of autogenous cancellous bone grafts. This paper describes an innovative technique to fabricate 3D scaffolds with both precise micro-architecture and surface micro-textures designed to provide osteoconductive stimuli to CTPs and subsequent bone regeneration. This process allowed the fabrication of a 3D PDMS Texture scaffold with 66% porosity by volume that consisted of 300 μm diameter meandering vertical pores, 200 μm×400 μm horizontal pores, and 71% of the surfaces within the scaffold covered with 10 μm diameter and 10 μm high posts. These geometrical features allowed cells to attach, migrate, and proliferate; forming significantly bigger colonies (499 cells/colony) compared to scaffolds with smooth surfaces (188 cells/colony), that expanded in both vertical (1-4 PDMS layers deep) and horizontal (up to about 3 mm long) directions. These results support the hypothesis that the scaffold micro-architecture and surface micro-textures significantly affect cell behavior. Specifically, the scaffold micro-architecture allowed cells to migrate in three dimensions, while the 10 μm posts increased the cell number per colony on the 3D PDMS Texture compared to the 3D PDMS Smooth. Although the biocompatibility of PDMS permits in vitro experiments and the possibility of animal studies, it is desired to translate these fabrication processes to other implantable materials. Precise control of the micro-architecture and surface topography may provide bone scaffolds with a higher degree of osteoconduction. By engineering 3D scaffolds with optimum micro-architecture and surface micro-textures, it might be possible to control local tissue behavior, not only for bone, but also for a variety of other tissues.

From the above description of the invention, those skilled in the art will perceive improvements, changes and modifications. Such improvements, changes and modifications within the skill of the art are intended to be covered by the appended claims.

Having described the invention, we claim:

1. An implant comprising:
a plurality of parallel layers spaced apart by a plurality of members, each layer having a substantially uniform thickness between opposite surfaces and a plurality of openings that permit fluid flow through an interior of the implant defined by the layers, the surfaces of each layer including an array of micro-structures, each micro-structure having a substantially uniform shape and an average height of about 1 nm to about 20 μm.

2. The implant of claim 1, the openings in each of the layers having a substantially uniform diameter.

3. The implant of claim 2, the openings in each of the layers having a diameter of about 200 μm to about 900 μm.

4. The implant of claim 1, the array of micro-structures providing the surfaces of the layers with a microtexture effective to enhance cell growth and proliferation.

5. The implant of claim 1, each member comprising a column having a substantially uniform diameter and being provided in an array between the layers.

6. The implant of claim 5, the columns having an average height of about 100 μm to about 900 μm and an average diameter of about 50 μm to about 500 μm.

7. The implant of claim 6, each of the layers having a substantially uniform thickness.

8. The implant of claim 7, each of the layers having a thickness of about 5 μm to about 100 μm.

9. The implant of claim 7, the layers being formed from at least one of a ceramic material or a polymer material.

10. The implant of claim 9, the layers being formed from at least one of polydimethylsiloxane, poly-co-glycolic acid (PLGA), poly(lactide-co-glycolide), polycaprolactone, calcium phosphate, and hdroxyapatite.

11. The implant of claim 1, having a porosity of at least about 60% by volume of the implant.

12. The implant of claim 1, the implant having a surface area defined by the layers, the micro-structures being provided on at least about 70% of the surface area of the implant.

13. The implant of claim 1, the micro-structures comprising a plurality of posts, each post having a substantially uniform shape and extending substantially normal to the surfaces of the layers of the implant.

14. The implant of claim 11, the posts having a diameter of about 1 μm to about 40 μm.

15. The implant of claim 1, the micro-structures comprising a plurality of ridges, the ridges having a substantially uniform height and being aligned on each layer in a manner effective to form a plurality of channels on the surface of each layer.

16. The implant of claim 15, the channel having an average width of about 1 μm to about 10 μm.

17. A scaffold for tissue engineering applications comprising:
a plurality of parallel layers spaced apart by a plurality of members, each layer having a substantially uniform thickness between opposite surfaces and a plurality of openings that permit fluid flow through an interior of the scaffold defined by the layers, the surfaces of each layer including an array of micro-structures, each micro-structure having a substantially uniform shape and an average height of about 1 nm to about 20 μm.

18. The scaffold of claim 17, the array of micro-structures providing the surface of the layers with a micro-texture effective to enhance cell growth and proliferation.

19. The scaffold of claim 17, each member comprising a column having a substantially uniform diameter and being provided in an array between the layers.

20. The scaffold of claim 17, the layers being formed from at least one of a ceramic material or a polymer material.

21. The scaffold of claim 17, the layers being formed from at least one of polydimethylsiloxane, poly-co-glycolic acid (PLGA), poly(lactide-co-glycolide), polycaprolactone, calcium phosphate, and hdroxyapatite.

22. The scaffold of claim 17, the implant having a surface area defined by the layers, the micro-structures being provided on at least about 70% of the surface area of the implant.

23. The scaffold of claim 22, the micro-structures comprising a plurality of posts, each post having a substantially uniform shape and extending substantially normal to the surfaces of the layers.

24. The scaffold of claim 22, the micrcrostructures comprising a plurality of ridges, the ridges having a substantially uniform height and being aligned on each layer in a manner effective to form a plurality of channels on the surface of each layer.

25. The scaffold of claim 22, further comprising a plurality of cells seeded on the scaffold.

26. The scaffold of claim 25, the cells comprising connective tissue progenitor cells.

27. A method of forming an implant for tissue engineering;
providing a plurality of layers, each layer comprising a substantially uniform thickness between opposite surfaces, a plurality of members that extend from the layers, and a plurality of openings that extend through the layers, the surfaces of each layer including an array of micro-structures, each micro-structure having a substantially uniform shape and an average height of about 1 nm to about 20 μm;
bonding the layers together so that the layers are substantially parallel to and separated from each other by the plurality of members.

28. The method of claim 27, the array of micro-structures providing the surfaces of the layers with a microtexture effective to enhance cell growth and proliferation.

29. The method of claim 27, each member comprising a column having a substantially uniform diameter and being provided in an array between the bonded layers.

30. The method of claim 27, the layers being formed from at least one of a ceramic material or a polymer material.

31. The method of claim 27, the layers being formed from at least one of polydimethylsiloxane, poly-co-glycolic acid (PLGA), poly(lactide-co-glycolide), polycaprolactone, calcium phosphate, and hdroxyapatite.

32. The method of claim 27, the micro-structures comprising a plurality of posts, each post having substantially uniform shape and extending substantially normal to the surfaces of the layers.

33. The method of claim 27, the micrcrostructures comprising a plurality of ridges, the ridges having a substantially uniform height and being aligned on each layer in a manner effective to form a plurality of channels on the surfaces of each layer.

34. The method of claim 27, the layers being formed by molding a polymer between a first mold portion and a second mold portion.

35. The method of claim 34, the first mold portion and the second mold portion being formed using photolithography.

36. The method of claim 35, the first mold portion comprising a microtextured first mold surface and the second mold portion comprising a microtextured second mold surface, the microtextured first mold surface and the microtextured second mold surface being capable of forming the micro-structures in each layer.

37. The method of claim 27, further comprising seeding the bonded layers with a plurality of cells.

38. The method of claim 37, the cells comprising connective tissue progenitor cells.
